(19) United States
(12) Patent Application Publication
(10) Pub. No.: US 2018/0015297 A1
(43) Pub. Date: Jan. 18, 2018

(54) SYSTEMS AND METHODS FOR TREATING HEAD INJURY USING MULTI-COLOUR LIGHT

(71) Applicant: MEDITECH INTERNATIONAL INC., TORONTO (CA)

(72) Inventor: FRED KAHN, TORONTO (CA)

(73) Assignee: MEDITECH INTERNATIONAL INC., TORONTO (CA)

(21) Appl. No.: 15/647,651

(22) Filed: Jul. 12, 2017

Related U.S. Application Data
(60) Provisional application No. 62/361,824, filed on Jul. 13, 2016.

(51) Int. Cl. A61N 5/06; A61N 5/067

(52) U.S. Cl. CPC A61N 5/0613; A61N 2005/067; A61N 2005/0663; A61N 2005/0626; A61N 2005/0652; A61N 2005/0647; A61N 2005/0659

(57) ABSTRACT

A system that uses light to treat concussions includes: one or more light array devices that are flexible and emit red light and infrared light; a red laser probe; an infrared laser probe; and a computing system connected to the light array devices and the laser probes. The computing system includes a processor, memory, a display device and a user input device. The memory includes multiple protocols, each protocol including multiple stages, and each stage including multiple steps for using the light array devices and the laser probes. The steps are respectively associated with predetermined settings for configuring the light array devices and the laser probes. The processor determines a given stage of a given protocol, and automatically configures settings of the light array devices and the laser probes in sequence according to the predetermined settings respectively associated with given steps within the given stage.

### CROSS-REFERENCE TO RELATED APPLICATIONS

This application claims priority from U.S. Patent Application No. 62/361,824 filed on Jul. 13, 2016, and titled “Systems and Methods for Treating Head Injury Using Multi-Colour Light”, the entire contents of which are herein incorporated by reference.

### TECHNICAL FIELD

The following relates generally to treating an injury with light.

### BACKGROUND

Light treatment of patients for various medical conditions is well known. Light treatment of injuries such as sport injuries and sprains as well as chronic conditions such as arthritis, sciatica, and chronic slow healing wounds or sores, are all well known.

The principle of all these light treatments is the targeted application of light to the area of the patient's condition (also referred to as pathology). It is found that in order to be effective, the light source should be in close contact with the skin. The light source is usually an array or panel of light emitting diodes, or in some cases low level laser. The treatment typically becomes more effective over longer periods. The light sources may, for example, be left in contact with the skin and the light rays penetrate into the tissues. This has been found to be efficacious in many instances. Other forms of light treatment include positioning a light source facing the skin, but not in contact with the skin.

### BRIEF DESCRIPTION OF THE DRAWINGS

Embodiments will now be described by way of example only with reference to the appended drawings wherein:

FIG. 1 is a block diagram of an example computing system in data communication with multiple light treatment devices.

FIG. 2 is a block diagram of another example computing system in data communication with multiple light treatment devices.

FIG. 3 is a perspective view of a computing system, two light treatment arrays and two laser probes.

FIG. 4 is a block diagram of an example light treatment array device.

FIG. 5 is an example configuration of light emitting elements arranged in an array on a light treatment array device.

FIG. 6 is an example configuration of traces on an opposite side the light treatment array device shown in FIG. 5.

FIG. 7 is an example of another light treatment array device which covers the head, neck, shoulders and upper back of a person.

FIG. 8 is a perspective view of the light treatment array device shown in FIG. 7, but in isolation.

FIG. 9A is a side view of an example laser probe.

FIG. 9B is a partial cross-sectional view of the laser probe shown in FIG. 9A.

FIGS. 9C and 9D are illustrations of a human body indicating the areas to place the laser probe for treatment. The laser probes can also be placed in each individual nostril (not shown).

FIG. 10 is an example of computer executable instructions for automatically determining a light treatment protocol and controlling the light treatment devices according to the determined protocol.

FIG. 11 is an example of settings and images stored by the computing system.

FIG. 12 is an example of computer executable instructions for modifying the last used treatment stage.

FIG. 13 are examples of protocols stored in the computing system based on different areas.

FIGS. 14A and 14B show an example of computer executable instructions for obtaining light treatment device settings and configuring the light treatment devices according to the settings.

FIGS. 15, 16, 17 and 18 are examples of images associated with tags, which are displayed as part of the GUI to guide placement of the light treatment devices.

FIG. 19 is an example of computer executable instructions for using a light treatment device which covers the head, the neck, and the shoulders.

FIGS. 20(a), 20(b) and 20(c) are different views of an example embodiment of a light array device in the form of a helmet.

### DETAILED DESCRIPTION

It will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures and components have not been described in detail so as not to obscure the example embodiments described herein. Also, the description is not to be considered as limiting the scope of the example embodiments described herein.

It is herein recognized that a head injury may have prolonged undesirable effects to the person who suffered from the head injury. In particular, it is herein recognized that post concussion, complex and varying symptoms resulting from both biological and neurochemical changes resulting from the injury may persist often for months and years. Depending on how long ago the injury was sustained, cases are classified as either acute (e.g. the concussion occurred less than a year ago) or chronic (e.g. the concussion occurred more than a year ago). Often, patients have been traumatized by time, the severity of the brain damage and a prolonged history of previously failed treatments, resulting in psychological aberrations including depression, labile mood swings and a high level of sensitivity to environmental and other factors. Therefore, depending on the psychological state and how fragile and sensitive the patient is (labile or stable), a further categorization is added, resulting in the following three clinical classes:

1. Acute/labile

2. Acute/stable

3. Chronic stable (e.g. applied to the average patient who is somewhat habituated to symptoms)

It is herein recognized that typical devices and methods of treating head injuries, such as a concussion, may not be effective or may require invasive techniques. Other approaches may be time consuming and difficult to implement.

It is also herein recognized that different treatment devices are used to treat head injury and that these treatment devices are operated and controlled in different ways. Furthermore, different machines are typically used to control these different devices, which can be troublesome. Therefore, it is herein recognized that it is desirable for a single control device to control the different treatment devices in a coordinated manner. It is also herein recognized to provide a unified electronic user interface to control the different treatment devices in a coordinated manner.

It is also herein recognized that typical devices and methods for treating concussions may be generic, and do not recognize that different patients have different degrees and types of trauma. Therefore, typical devices and methods for treating concussions do not take a careful and individualized approach based on assessment data.

It also herein recognized that, from the perspective of a clinician, typical devices and methods may require many steps and settings that are adjusted and performed by a clinician or user. Steps may be forgotten or performed in the wrong order. Furthermore, the device may be adjusted to the incorrect settings for a patient. The potential for error is increased when the same device is used for many different patients, which often occurs in a clinic environment.

The proposed system and methods address one or more of the above issues. In particular, the proposed system and methods described herein provide a computing apparatus, also herein called a computing system, and various light treatment devices to treat concussion. The various light treatment devices are connectable to the multiple ports of the computing apparatus, and the computing apparatus controls these various light treatment devices in a coordinated manner that is specific to a given patient. Based on data related to a given patient, treatment protocols are automatically selected and the light treatment devices are automatically configured to certain settings. The computing apparatus displays, via a display device, a graphical user interface that displays prompts to guide an operator (e.g. a clinician) through the treatment protocol in conjunction with the settings of the one or more light treatment devices. In other words, a single computing apparatus is configured to be connected to, and to control, multiple various light treatment devices according the principles described herein.

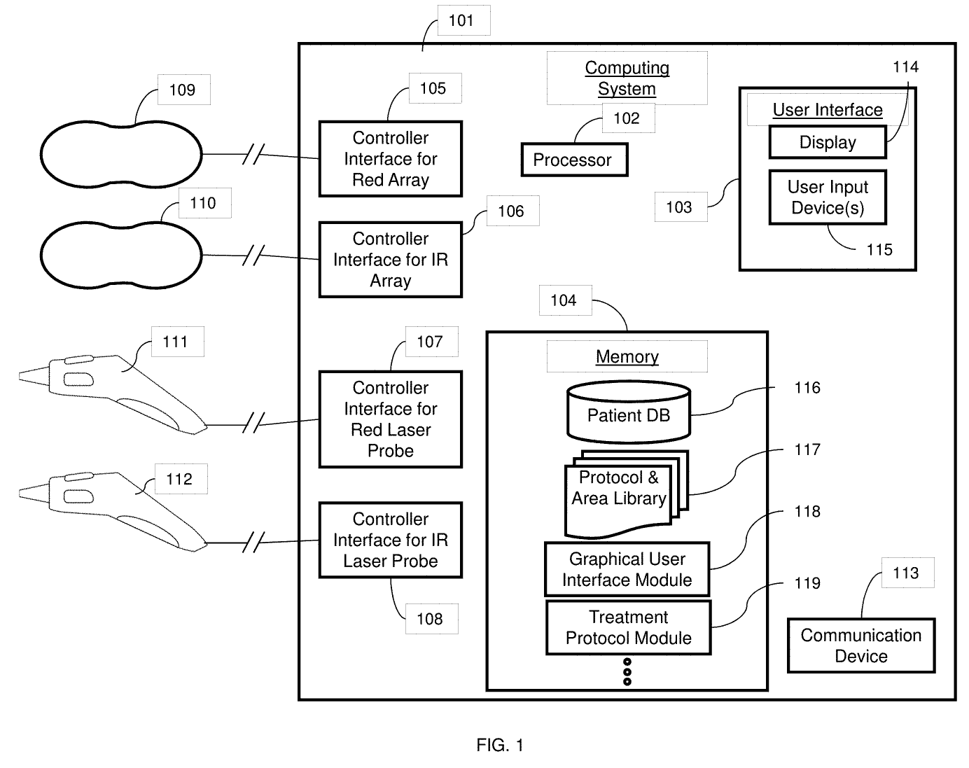

Turning to FIG. 1, an example embodiment of the computing apparatus, or computing system, 101 is shown connection with a red light array device 109, an infrared light array device 110, a red laser device or red laser probe 111, and an infrared laser device or infrared laser probe 112. It will be appreciated that the term “infrared” will herein be referred to as IR.

The light treatment devices 109, 110, 111, 112 can be oriented and located onto the skin of patient and is operable to emit light from one or more light sources incorporated into each device onto a desired area of the patient at one or more wavelengths. The light treatment devices may also be referred to as treatment heads.

The shapes of the light treatment devices 109, 110, 111 and 112 are for example, and it will be appreciated that other shapes and configurations of light treatment devices that are consistent with the principles described herein may be used.

The computing system 101 includes a processor 102, a user interface 103, memory 104 and a communication device 113. The communication device is configured for wireless data communication in an example embodiment. The communication device is configured for wired data communication in another example embodiment.

The computing system further includes a controller interface 105 for the red light array 109, a controller interface 106 for the IR array 110, a controller interface 107 for the red laser probe 111, and a controller interface 108 for the IR laser probe 112. For example, these interfaces include connection ports located on the housing of the computing system, so that wires of these different devices can plug into the computing system via these connection ports.

The user interface 103 includes a display device 114 and one or more user input devices 115. For example, the user input devices include one or more physical buttons incorporated into the computing system and a touch-sensitive screen incorporated into the display device 114. In other example embodiments, the user input devices also include a mouse and a keyboard. In other example embodiments, the user interface 103 includes a speaker, one or more indicator lights, a microphone, or various other input and output devices known in the art. It will be appreciated that other input devices for interacting with a computing systems are applicable to the principles described herein.

The memory 104 includes a patient database 116, a protocols and areas library 117, a graphical user interface (GUI) module 118 and a treatment protocol module 119. It will be appreciated that there may be other modules.

The patient database 116 includes information about different patients. For example, for a given patient, information stored in the database includes data specifying the type of concussion, associated treatment history and status using the light treatment system, and progress related to the light treatment. In particular, the associated treatment history and status using the light treatment system includes the previously performed stages of treatment for a given protocol and a given area of the patient (e.g. on the head, neck, shoulders, etc.) and the associated dates of these previous treatments. The patient database 116 may also include proposed next light treatments for the given patient that are based at least on the associated treatment history and status.

The protocols and areas library 117 includes different light treatment protocols associated with different areas on a person's body for treating a concussion. The selection of protocols is based on the data in the patient database 116. In an example embodiment, the selection or determination of a protocol for a treatment is automatically made by the computing system 101. In an example embodiment, the selection or determination of a protocol for a treatment is automatically assisted by the computing system 101.

A protocol herein refers to a sequence of steps to treat a patient for a concussion using the light treatment devices. A protocol includes the order of when to activate the light treatment devices, positioning or a specification of an area of where to place a given light treatment device on the patient, the length of time to apply the light on the specified area, and the operation settings of the given light treatment device. The implementation of the selected protocol includes computer executable instructions that automatically control parameters of timing, frequency, power, etc. associated with the one or more light treatment devices.

The GUI module 118 is used to receive inputs from a user (e.g. the operator) via the user interface 103 about a given patient, and to display information about the operation status of the light treatment devices. The GUI module enables a user to control the light source or light sources on a given light treatment device and to receive data from the given light treatment device via one or more of the interface controllers 105, 106, 107 and 108. This data may include, for example, the operational status of the treatment head (e.g. to determine whether the light treatment device is in an operating condition), the operational parameters of the light treatment device (e.g. the wavelength(s) and waveform at which the light source is emitting), the temperature of the light treatment device, and other information relevant to an operator of the light treatment device.

The GUI module 118 also provides prompts via the user interface 103 (e.g. visual prompts or audio prompts, or both) to the operator regarding when and where to place a given light treatment device according to the prescribed protocol.

The treatment protocol module 119 is configured to automatically select a protocol from the library 117, or, in another embodiment, to assist an operator in selecting a protocol from the library 117. The treatment protocol module 119 also generates commands to automatically configure the settings of the light treatment devices consistent with the specified protocol. The module 119 also sends commands to activate and deactivate a given light treatment device for a time period that is part of the specified protocol. The commands affecting the light treatment devices are sent to one or more of the appropriate controller interfaces 105, 106, 107, and 108. For example, a command to control the red laser probe 111 is sent to the controller interface 107.

Each controller interface module is operable to control various parameters of the light sources or light source on a given light treatment device including, for example, the intensity of emitted light, the duration of light emission, the number of cycles of treatment to be applied to a particular area of the patient, and the wavelength of light emitted onto the patient. Specific values for these settings are part of a given protocol.

Each controller interface module includes, or is linked to, a power source which powers the light source module(s) on a given light treatment device. In another example, a given light treatment device (e.g. 109, 110, 111, 112) otherwise, or in addition, includes an on-board power source such as a battery to power the light source module(s).

In an example embodiment, the computing system 101 has a housing that provides one convenient apparatus to control the various light treatment devices 109, 110, 111, 112. It also provides a user interface 103 that provides unified information and control over the various light treatment devices in relation to a given patient. This improves the efficiency of this light therapy system, improves the accuracy of the control of the light treatment devices in relation to a given patient, and improves the ease at which a person can use the light therapy system.

Additional details about the operation of the light treatment devices according to the protocols are provided below.

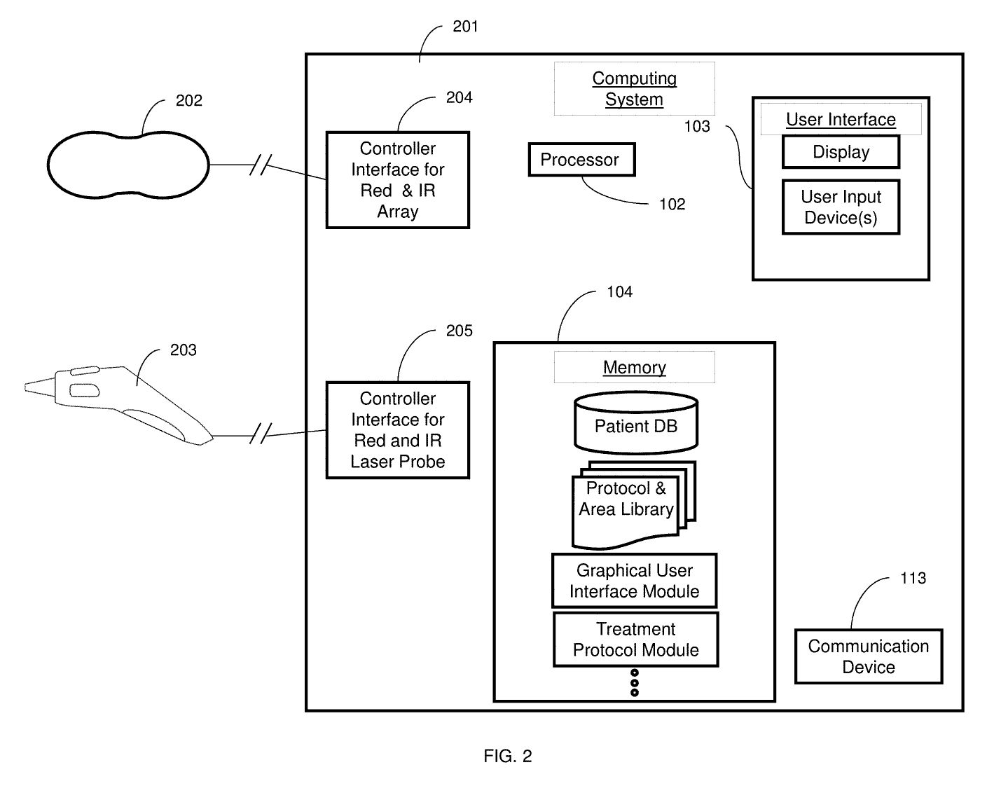

Turning to FIG. 2, another example embodiment of a computing system 201 is shown connected to a light array device 202 (also a light treatment device) and a laser probe device 203 (also a light treatment device). The light array device 202, also called a combined light array, is configured to radiate both red light and IR light at separate times. The laser probe 203, also called a combined laser probe, is configured to radiate both red laser and IR laser at separate times. The controller interface 204 controls the functionality of both the red light and the IR light produced by the combined light array 202. The controller interface 205 controls the functionality for both the red laser and the IR laser produced by the combined laser probe 205.

The devices 202 and 203 reduce the number of components in the system and reduce the steps for an operator to switch between different devices.

It will be appreciated that the numeral 101 is hereon used to reference the computing system and may, depending on the application, refer to the configuration of the computing system 201 shown in FIG. 2.

It will be appreciated that in another example embodiment, not shown, the computing system is connected to the combined light array device 202, a red laser probe 111 and an IR laser probe 112. In another example embodiment, not shown, the computing system is connected to the combined laser probe 203, a red light array 109 and an IR light array 110. It will be appreciated that different combinations of the devices may be used to provide red light, IR light, red laser and IR laser.

It will be appreciated that the different light treatment devices can be connected to the computing system 101 depending on the treatment protocol.

Although not shown, in an example embodiment, the communication device 113 is used to communicate with other computing devices or servers. For example, other computing devices or servers in a hospital or health clinic may include patient data and this patient data is transmitted to the computing system 101, so that the patient data is stored in the patient database 116. In another example, data obtained or generated about a patient's light treatment for their concussion on the computing system 101 is shared with other computing devices, such as other computing devices in the hospital or the health clinic.

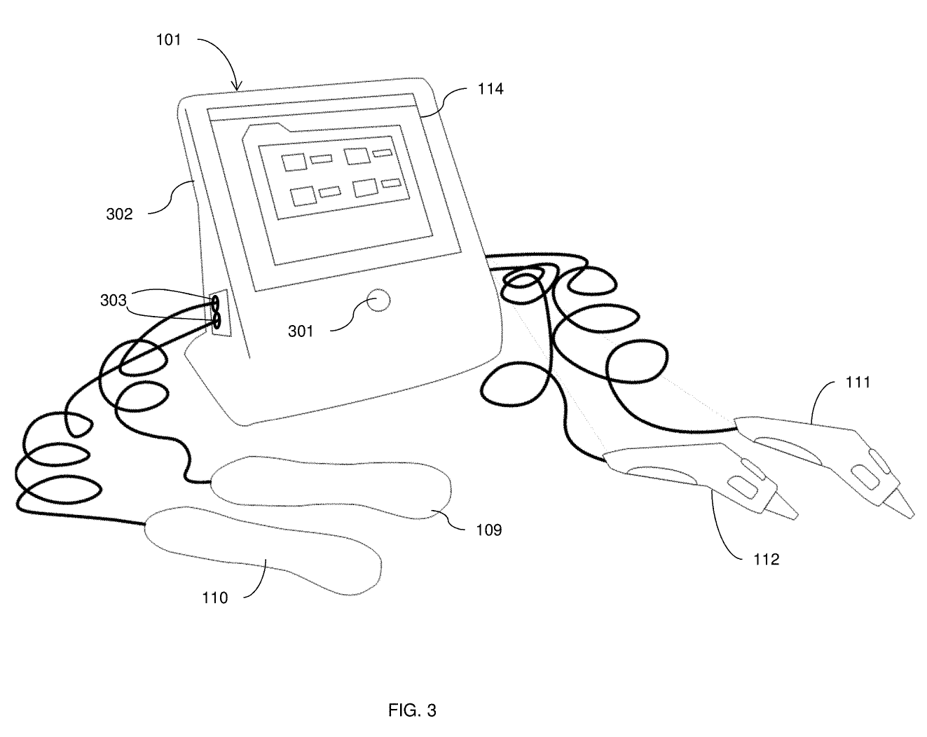

Turning to FIG. 3, an example embodiment of the housing 302 of the computing system 101 is shown connected to the light treatment devices 109, 110, 111 and 112. In the example shown, the light treatment devices are connected to the computing system via wires. The wires are plugged into connection ports 303 located on the housing of the computing system.

The display device 114 is also shown. Preferably, although not necessarily, the display 114 is a touch screen.

Physical buttons may be located on the computing system 101. An example button 301 is shown on the front face of the body of the computing system.

It will be appreciated that other shapes and configurations of the housing of the computing system may be used according the principles described herein.

In an example embodiment, the light treatment array devices 109, 110, 202 are flexible light treatment pads that are configured for attachment and/or positioning on a patient for receiving light treatment therapy.

The light treatment array devices are substantially flexible for being positioned on and for conforming to the shape and curvatures of the patient's body that is receiving the light treatment.

It will be appreciated that any module or component exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the computing system 101 or any one of the light treatment devices (e.g. light treatment array device or laser probe device) described herein or accessible or connectable thereto. Any application or module herein described may be implemented using computer readable/executable instructions that may be stored or otherwise held by such computer readable media.

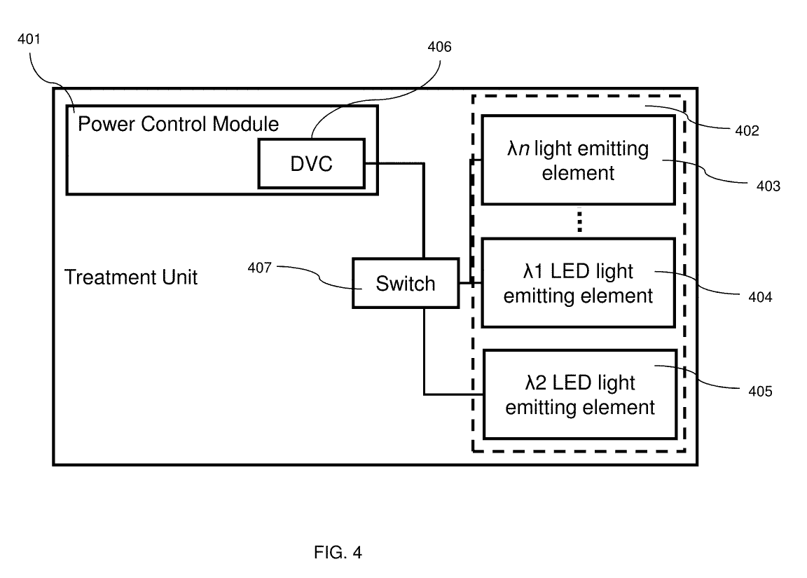

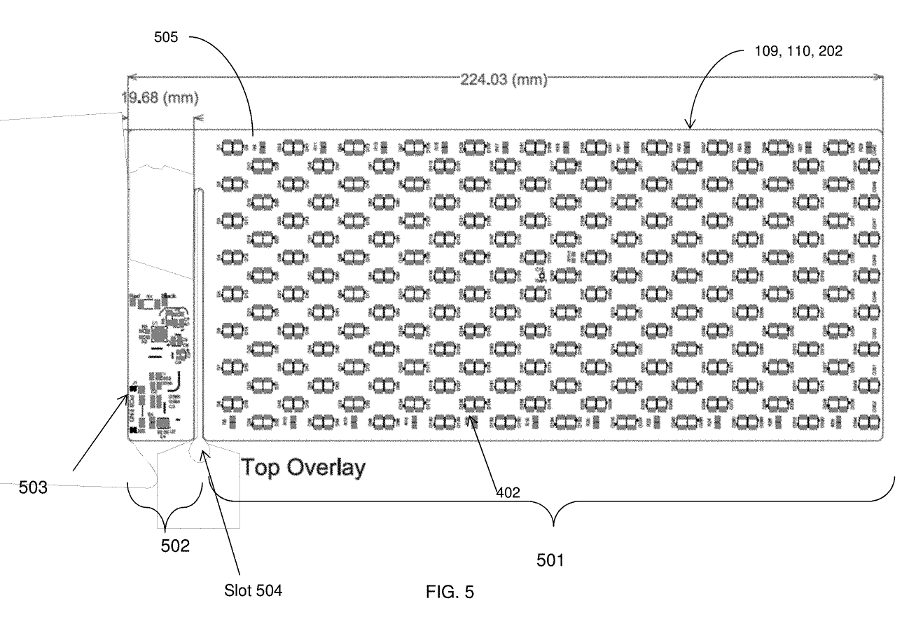

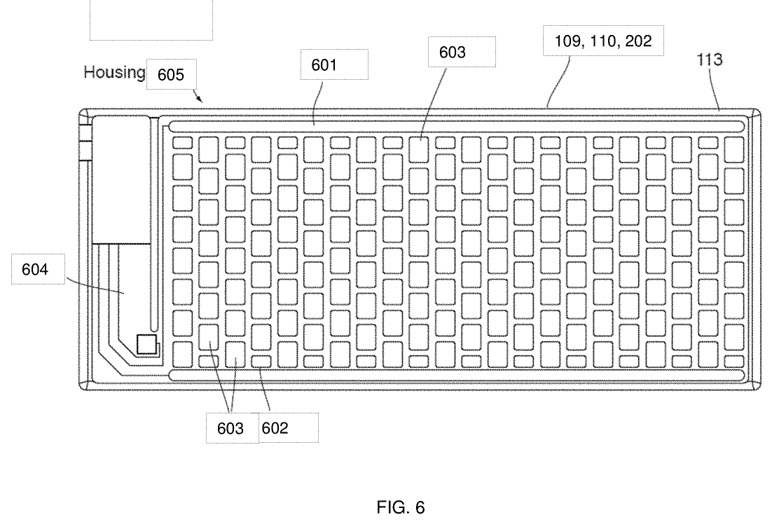

Turning to FIG. 4, an example embodiment of components in a given light array device (e.g. 109, 110, 202) is shown. A light treatment array device includes a power control module 401 and one or multiple light source modules 402. The light source modules 402 are interconnected electrically on a flexible circuit board 601 (see FIG. 6) and disposed on one side of the board 601. The modules 402 are constrained to a first portion 501 of the light array device (see FIG. 5). The power control module 401 is mounted on a second portion 502 of the board 601 and controls power delivered to one or more of the light source modules 402. In one embodiment, the light modules 402 may be an array of LED's emitting a single wavelength, or arrays of bi-color LED's. In each case, a suitable power control module 401 controls power to the arrays in response to signals from the corresponding controller interface.

The power control module 401 is operable to power a dynamic voltage controller (DVC) 406, which is operable to apply a driving voltage to first, second, and nth LED light emitting elements 404, 405, and 403 in the light source modules 403 via the switch 407.

A connector interface 503 (see FIG. 5) is provided on the second portion 502 of the board 601 for coupling the light treatment array device to the computing system 101. It is appreciated that the computing system 101 transmits powers and control data to the light treatment array device via the interface 503. In an example embodiment, the light treatment array device sends feedback data to the computing system 101 via the interface 503.

A given light treatment array device (e.g. 109, 110, 202) is configured to receive one or more control signals from the computing system 101 for causing the emission of light from one or more of the light source modules 402 onto a desired area of the patient's body at one or more predefined wavelength(s).

Each light source module 402 comprises one or more light emitting elements (indicated at 404, 405 and 403 in FIG. 4). The light emitting elements includes light emitting diodes (LED). In another example embodiment, the light emitting elements may comprise LASER, and in another example embodiment the light emitting elements include a combination of LED and LASER. It will be appreciated that the light emitting elements will be chosen to provide the wavelength and type of radiation required to produce the desired therapeutic effect.

In one example embodiment, arrays of light emitting elements 404, 405, 403 of different wavelengths λ1, λ2, λ3 respectively, are used to create a multi-colour light treatment array device. For example, the light treatment array device includes an array of LEDs, a first group of which comprise light emitting element 404 and emit light at a wavelength correspond to red light (e.g. in the range of 620-750 nm) while a second group comprising light emitting element 405 and emit light at a wavelength corresponding to IR light (e.g. in the range of 1 nm to 700 nm). In an example embodiment, one light emitting elements emits light at 660 nm and the other light emitting element emits light at 840 nm. The LEDs are, for example, arranged in an array such that the LEDs 404 of the first wavelength λ1 are substantially evenly distributed throughout the light treatment array device and the LEDs 405 of the second wavelength λ2 are also substantially evenly distributed throughout the light treatment array device and interposed between the LED's 404.

Referring to FIGS. 5 and 6, each light emitting element 404, 405, 403 has a pair of conductors, 602, each of which is electrically connected to a respective connector pad 603. The connector pads 603 are disposed on the opposite side of the board to the modules 402, so as to be directed in the opposite direction to the modules 402. Preferably, the connector pads 603 occupy a larger surface area than the light emitting elements (e.g. 404 and 405) with a minimal gap between each of the connector pads 603. The major portion of side 604 of the circuit board 601 is thus occupied with the connector pads 603 in order to achieve a larger surface area for effective heat dissipation and act as a heat sink body. As shown in FIG. 6, the connectors 603 are square and arranged linearly to coincide with the array of the light emitting elements (e.g. 404, 405, 403). Where a different array is required, alternative shapes may be provided, such as hexagonal, triangular or rectangular to give a nested arrangement covering one of the major surface of the board 601.

The shape and configuration of the connector pads 603, which advantageously are made of copper for good heat and electrical conductivity, is selected to maximize the surface area of the connector pads 603 relative to the surface area of the circuit board 601 while forming the electrical conductive traces on the circuit board 601. The flexible circuit board 601 is preferably made from any one or more of the following materials: polyimide copper clad with or without adhesive, polyester copper clad or any flexible film or metal clad.

Referring again to FIG. 5, the first and the second portions 501 and 502 of the board 611 are mechanically isolated from each other by a discontinuity or slot 504. The slot 504 extends across the board 601 from one edge and terminates prior to the opposite edge to provide a bridge 505 between the two portions 501 and 502. The bridge 505 provides an area of the board 601 that allows electrical connectivity between the first and the second portion 501 and 502. The extent of the slot 504 will vary depending on the flexibility required and the connections to be made between the two portions. In general, the slot will extend as far as practical without jeopardising the structural integrity of the board 601.

The second portion 502 supports or incorporates the generally inflexible or rigid circuitry components (e.g. drivers, communication interfaces, control circuitry, switches) of the light treatment array device. The slot 504 provides a partition for physically separating or isolating the flexible from the inflexible circuitry components on the light treatment array device. In this manner, the first portion 501 is in contact (e.g. placed on a surface for receiving treatment) with the patient for providing treatment is able to flex to contour to the surface area to be treated while the second portion 502 that does not need to be in direct contact with the patient's skin and so does not need to flex, is not subjected to the strains imparted by such flexure. The first portion 501 is thus able to flex along a number of different axes to conform to the shape of the patient's skin, and conform to highly contoured areas of the patient, such as the neck or the head.

The flexible circuit board 601 is encased within a flexible housing 605 made from an insulating material that is optically transparent to the wavelength of the radiation emitted by the modules 402. The insulating material is preferably a high consistency silicone rubber. Generally, the moulding layers forming the housing 605 can be selected from the group consisting of: silicone, thermoplastic, polyurethane, and thermoplastic elastomer (TPE).

The separation of the inflexible components in the second portion 502 from the flexible components in the first portion 501 by the slot 504 allows a high degree of pliability and flexion in the overall light treatment array device as the second portion 502 occupies a substantially smaller surface area than the first portion 501. Additionally, by separating the flexible from the inflexible circuit components, this provides a stress relief to the relatively rigid components mounted on the light treatment array device during flexure and bending for conforming to various body contours and reduces the likelihood of damage to the light treatment array device components.

A strap may be included to secure the light treatment array device to a patient's body (e.g. neck, head, or shoulders).

Additional details of an example embodiment of a light treatment array device, also called treatment pad, is described in U.S. patent application Ser. No. 14/690,768 filed on Apr. 20, 2015, the contents of which are herein incorporated by reference. Another example of a light treatment array device is shown in U.S. patent application Ser. No. 13/355,162 filed on Jan. 20, 2012, the contents of which are incorporated herein by reference

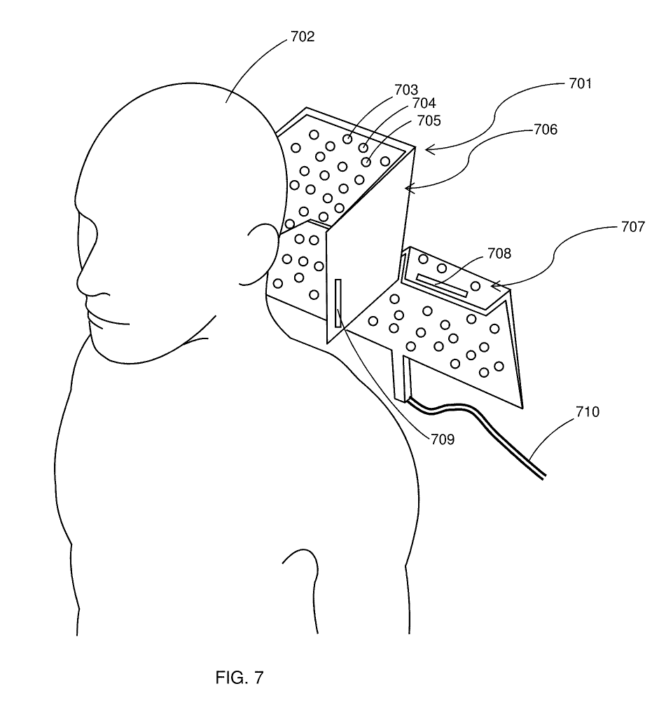

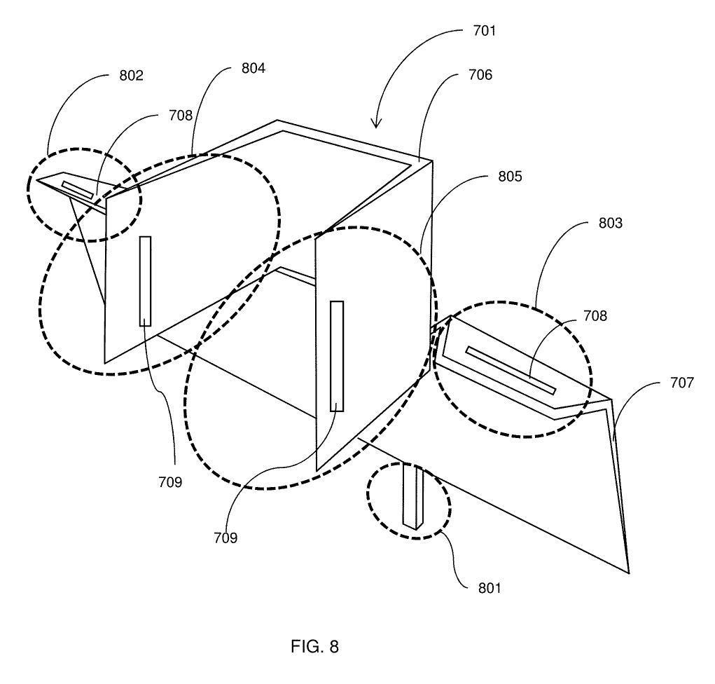

Turning to FIGS. 7 and 8, another example embodiment of a light treatment array device 701 is shown, which has generally the same functionality and similar components as the light treatment array device described above. The design of the shape or form of the device 701 is however different than the shape or form of the devices 109, 110 and 202. The device 701 is connectable to the computing system by a wire 710, and the device 701 is controllable by the computing system.

The device 701 covers a large surface including the back of the head of a person 702, the neck, and the upper back. The device 701 includes two flexible portions 706 and 707. The neck and head portion 706 of the device 701 is positioned above the upper back portion 707 of the device 701. The neck and head portion, or the first portion, 706 is able to flex around both sides of the neck and the head. In particular wings 804 and 805 on opposite ends protrude towards the sides of the neck and head portion 706 to cover sides of a person's neck and head. The upper back portion 707 is able to flex to the contours of the back of the person 702 and above the shoulders of the person. In particular, wings 802 and 803 on opposite ends protrude from the sides of the upper back portion 707.

It will be appreciated that the portions 706 and 707 are connected. In an example embodiment, portions 706 and 706 are integrally formed to form a single device. The portions 706 and 707 are able to flex relative to each other to confirm to the curvature between the neck and the back of a person.

It will be appreciated that other designs for the shape of the device 701 are applicable to the principles described herein. The design of the device 701 shown in FIGS. 7 and 8 is not dictated solely by a utilitarian function. The shape of the device 701 shown in FIGS. 7 and 8 has aesthetic appeal.

At least the majority of one of the major surfaces of the device 701 is covered with an array of light emitting sources 703, 704 and 705. Different light emitting sources may emit light at different wavelengths, such as red light or IR light.

The light emitting sources are grouped based on the areas located on the device 701. For example, one group of light emitting sources is located in the middle area of the head and neck portion 706; another group of light emitting sources is located on the left area of the head and neck portion 706 (e.g. the left side wing 804); another group of light emitting sources is located on the right area of the head and neck portion 706 (e.g. the right side wing 805); another group of light emitting sources is located in the middle area of the upper back portion 707; another group of light emitting sources is located on the left area of the upper back portion 707 (e.g. includes the left side wing 802); and another group of light emitting sources is located on the right area of the upper back portion 707 (e.g. includes the right side wing 803). Other groupings of light emitting sources are applicable to the principles described herein.

In an example embodiment, a single group of the light emitting sources may be activated one at a time to radiate light one area on the body of the person. In another example embodiment, selected multiple groups of light emitting sources may be activated simultaneously to radiate light on a larger area or different areas on the body of the person. In an example embodiment, the computing system 101 automatically controls which grouping of light sources are activated according to a predetermined sequence and a predetermined time.

For example, a first group of light sources at a first location is activated for X seconds and according to a first set of parameters, and then subsequently or simultaneously, a second group of light sources at a second location is activated for Y seconds and according to a second set of parameters. The first and the second locations are different. In this way, the device 701 can treat different areas of a patient without having to remove or reposition the device on the patient.

Slots 708 and 709 are also included on the device 701 for receiving a strap. One or more straps are used to secure the device to the body of a person. It will be appreciated that the position of the straps and the shape of the straps may be different according other example embodiments. The position and the shape of the straps shown in FIGS. 7 and 8 have aesthetic appeal.

In an example embodiment, a lower portion 801 of the device 701 is similar to the second portion 502 described above. The lower portion 801, in an example embodiment, houses inflexible or rigid circuitry components (e.g. drivers, communication interfaces, control circuitry, switches) of the light treatment array device 701.

In another example embodiment, the device 701 is built from two separate sections, such as the upper section 706 and lower section 707, and are combined together to form the device.

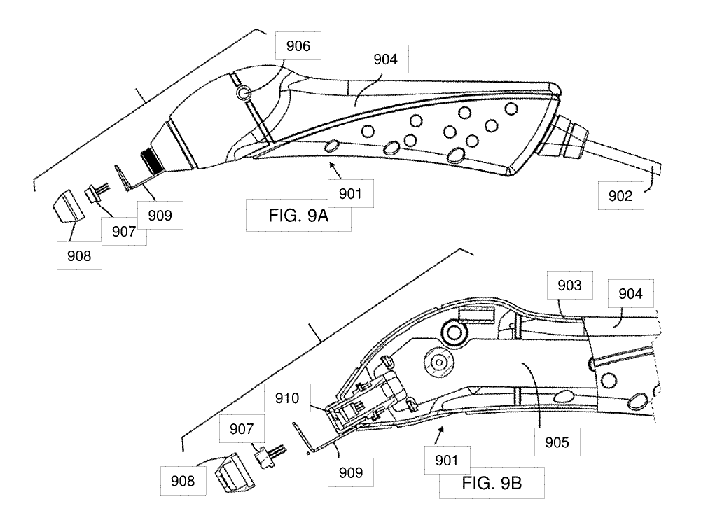

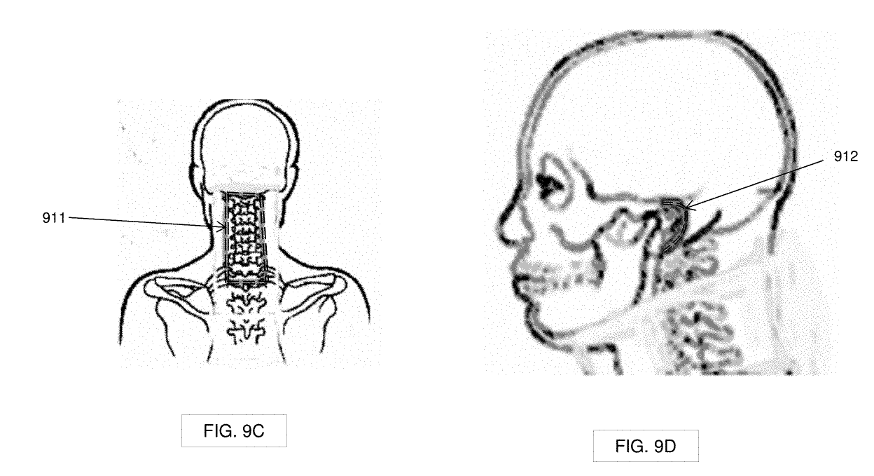

Turning to FIG. 9A and 9B, a laser probe 901, having a cord 902 is adapted to be connected to the computing system 101.

The laser probe is of the type that incorporates a single laser light treatment source, in this case typically a class 3 laser, or other forms of lighting source including laser light may be provided.

The probe is illustrated in more detail in FIG. 9B, and will be seen to include a right side panel 903 and left side panel 904, which may be mated together to form an enclosure. Typically the enclosure will be sized to fit conveniently in the hand of an operator, and may be about the size of a flashlight, substantially as shown, this being merely one particularly convenient example. Other shapes will suggest themselves to persons skilled in the art for various reasons, and the invention is not restricted to such shape. Within the side panels, there is a circuit board 905, on which is mounted a miniature CPU, and various electronic components such as are well known in the art. Alternatively there could be a mode control, for connecting to a variety of different mode programs within the device.

A control button 906 is positioned on the probe. Other button placements and shapes may be used. The button(s) extend through suitable openings in a side panel of the probe 901 for easy access. The laser probe is not specifically limited to any unique sequence of functions, and various different functions may be incorporated.

The probe is provided with an electrical cord 902 at one end. At the other end, the probe is provided with a treatment light assembly, comprising a treatment light socket 910, a light source, in this case a low level laser light source 907, and a protective cap 908. A spring wire connection 909 is provided, for connecting with the protective cap. The light source is provide with a plurality of contacts, which contact internal circuits (not shown) for supplying power from the circuit board to the light source.

The socket is of generally cylindrical shape, and is received within a recess, formed in the opposite left and right side panels. The protective cap 908 is formed of metal, and is connected by connection 909 to control circuits on the board 905. The cap 908 when contacted on the skin, functions like the buttons of an elevator, for example. The skin contact is sensed by the control circuits on the board 905 and switches on power to the light source 907. The purpose of this is to ensure that unless and until the protective cap 908 is pressed on the skin, the light source will not be activated.

When the protective cap 908 is pressed on the skin, the control circuits will activate the light source 907. Treatment will then be applied to that area of the body to which the probe is directed and applied. The moment the probe is lifted off the skin, contact to the light source is then broken, and no light will be emitted.

In an example embodiment, when the protective cap 908 is pressed on the skin, the probe emits laser light according to the settings automatically selected by the given protocol.

The laser probe shown in FIGS. 9A and 9B is an example applicable to the red laser probe and the infrared laser probe. Other configurations of probes that emit laser may be used according to the principles described herein.

FIGS. 9C and D show some of the different areas 911 and 912 in which the laser probes are placed according to acupoints. The laser probes can also be placed in each individual nostril (not shown). Other areas for placing the laser probes are described below.

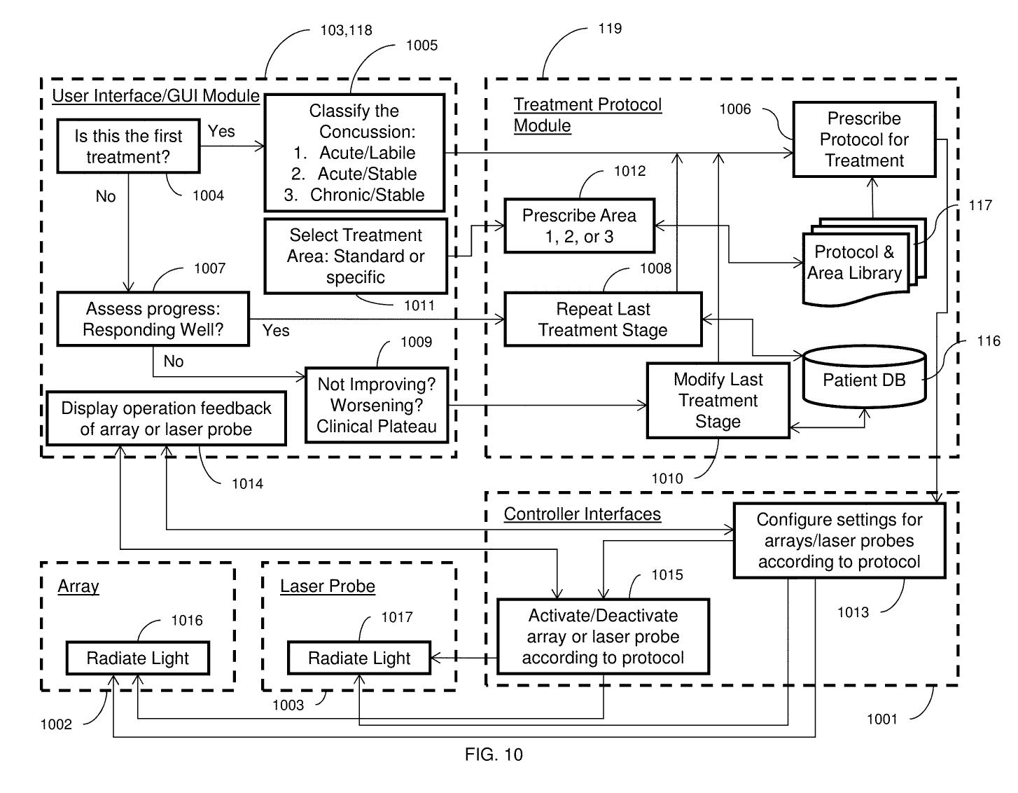

Turning to FIG. 10, example executable instructions are provided, which are performed by the computing system 101 and a given light treatment array device 1002 as well as a given laser probe device 1003.

Through the user interface 103 and GUI module 118, the computing system 101 confirms with the operator (e.g. a person) whether or not this session is the first treatment for a given patient (block 1004). This may also be determined automatically by the computing system 101 searching the patient database 117 to determine if the given patient has had previous treatments. If there are no previous treatments, then the computing system 101 determines that this session is the first treatment for the given patient. Otherwise, this session is not the first treatment.

If this session is the first treatment, then at block 1005, the GUI prompts the operator via the display device to enter in information that classifies the concussions as one of the following options: 1) acute/labile; 2) acute/stable; or 3) chronic/stable. The operator inputs the information or selection using the user interface 103. The selected classification is sent to the block 1006, in which the treatment protocol module 119 prescribes a protocol for treatment.

For first time treatments, the GUI displays prompts to the user via the display device to select a treatment area (block 1011). In an example embodiment, the display device displays the options of: 1) standard and 2) specific. The input from the user with respect to the area is used by the module 119 to automatically determine a specific treatment area on the patient's body (block 1012).

The prescribed areas are herein referred to as Area 1, Area 2 and Area 3. Details about each of the areas are stored in the protocol and area library 117. The prescribed area, which is outputted from block 1012, is used to inform the process of block 1006 to prescribe a protocol for treatment.

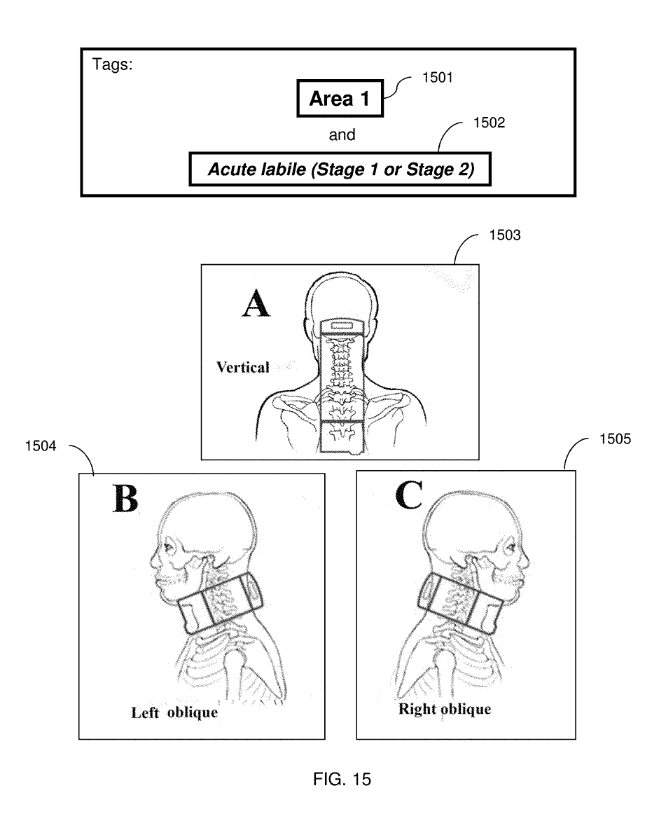

Below are general descriptions of each area in relation to an example treatment protocol, and FIGS. 15-18 include diagrams that illustrate the areas.

With respect to Area 1, it is herein recognized that in 70% of cases, red light and IR light followed by laser therapy applied over the cerebellum, brain stem, (including cranial nerve origins) and spinal cord, extending from the occipit to the T2 level vertically, and from C4 to T3 transversely are generally adequate to treat a patient who has had a concussion. Laser probes (e.g. red laser first followed by IR laser) are applied to the area of pathology (e.g. acupoints) extending from top of the cerebellum to T2 including the brain stem along the midline and paracervical tissues from C1-T2, including neurological structures, overlying soft tissues, nerve roots and skeletal structures.

With respect to Area 2, it is herein recognized that in up to 30% of patients, additional therapy is required over the cerebral hemispheres, beginning in the frontal area and extending to the upper margin of the cerebellum (e.g. Area 2).

With respect to Area 3, it is herein recognized that isolated injuries to the central nervous system are rare. It has become widely accepted that the brain stem, cranial nerve origins and spinal cord may also be involved, along with soft tissue and skeletal damage to the cervical spine. What is less well recognized by others, but is recognized herein, is that traumatic brain injury not infrequently extends to the ears. In particular, the middle ear containing the ossicles and the inner ear incorporating the semicircular canals are responsible for auditory acuity and equilibrium or body balance. Symptoms associated with these structures include loss of hearing, tinnitus, ringing and other unusual sensations, including ataxia. The loss of balance may be most pronounced. If several treatments of Area 1 and/or the central nervous system have not resolved the symptoms relating to the ear, protocols specific to the ear are utilized (e.g. Area 3). Laser probes are applied circumferentially around the external auditory canal and aim at the middle ear obliquely (e.g. Area 3).

The laser probes applied to the above areas are preferably positioned at acupoints, also called acupuncture points, within the above areas.

Continuing with FIG. 10, in an example embodiment, if the selected treatment area is “standard” (block 1011), then the prescribed area is Area 1. If the selected treatment area is specific then, the prescribed area for treatment is targeted. The operator will be able to specify the targeted location.

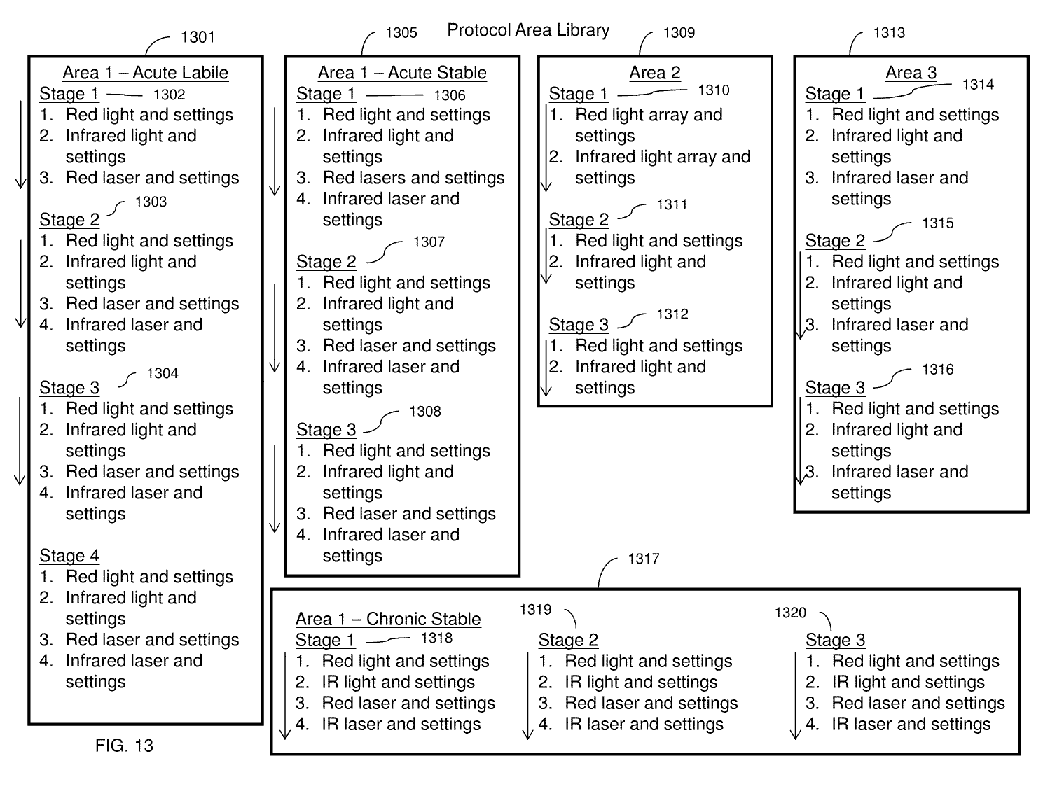

After obtaining the concussion classification and the prescribed area, the treatment protocol module uses this information to automatically determine a protocol for treatment (block 1006) which is obtained from the library 117. In particular, as shown in FIG. 13, if the classification is acute labile and the prescribed area is Area 1, then the treatment protocol module 119 automatically selects protocol 1301. If the classification is acute stable and the prescribed area is 1, then the treatment protocol module 119 automatically selects protocol 1305. If the classification is chronic stable and the prescribed are is Area 1, then the treatment protocol module 119 automatically selects protocol 1317. If the prescribed area is Area 2, then the treatment protocol module 119 automatically selects protocol 1309. If the prescribed area is Area 3, then the treatment protocol module 119 automatically selects protocol 1313. It will be appreciated that each of these protocols has multiple stages (e.g. Stage 1, Stage 2, and Stage 3) and that within each stage are multiple steps for applying different types of light therapy with certain light treatment devices configured at certain settings (e.g. frequency, duty cycle, duration of time, and power). Further details about these protocols are described below.

Continuing with FIG. 10, after automatically determining the protocol for the first treatment (block 1006), the treatment protocol module 119 sends commands to a controller interface 1001 corresponding to a certain light treatment device to configure settings of the certain light treatment device. The certain light treatment device depends on the protocol determined by the module 119 and the settings depend on the settings obtained from the library 117 that are associated with the protocol that has been determined by the module 119. It will also be appreciated that the controller interface 1001 is a representation of any one or more of the control interfaces 105, 106, 107, 108, 204 or 205 to assist in explaining the general process of FIG. 10. However, in a specific implementation, the command from the treatment protocol module 119 is sent to one or more of the control interfaces 105, 106, 107, 108, 204 or 205 depending on a given step within a given stage of the determined protocol.

For example, a first given step with a first given stage of a determined protocol includes activating red light emitting elements according to a first group of settings and therefore, a first command that includes the first group of settings is sent to the controller interface 105 for the red light array 109 or, in another embodiment, to the controller interface 204 for the combined red and IR light array 202. Continuing with the example, a second given step within the first given stage of the determined protocol includes activating IR light emitting elements according to a second group of settings and therefore, a second command that includes the second group of settings is sent to the controller interface 106 for the IR light array 110 or, in another embodiment, to the controller interface 204 for the combined red and IR light array 202. Continuing with the example, a third given step within the first given stage of the determined protocol includes activating a red laser emitting element according to a third group of settings and therefore, a third command that includes the third group of settings is sent to the controller interface 107 for the red laser probe 111 or, in another embodiment, to the controller interface 205 for the combined red and IR laser probe 203. It will therefore be appreciated that the commands sent to the general representation of controller interface 1001 and the specific controller interface depends on the step within a determined protocol.

After the controller interface 1001 receives the command and the associated settings, the controller interface configures settings for the light array device or the laser probe device according the received command and the associated settings (block 1013). The light array or the laser probe to be used in the step is displayed via the user interface 103 on the display device (block 1014).

After the controller interface 1001 configures the settings of the light array or the laser probe (block 1013), it activates the light array or the laser probe according to the protocol (block 1015). For example, if a red light array or an IR light array is activated, generally shown as array 1002 in FIG. 10, then responsive to the activation command, the array 1002 radiates light (block 1016) at the configured settings. In another example, if a red laser probe or an IR laser probe is activated, generally shown as laser probe 1017 in FIG. 10, then the laser probe 1017 radiates laser light (block 1017) at the configured settings. The activation status of the light array or the laser probe is displayed by the user interface 103 at block 1014.

After the duration time of the light or laser treatment is finished for a given step, the controller interface automatically turns off or deactivates the light array 1002 or the laser probe 1003.

The information about each completed step is stored in the patient database 116. Similarly, after each completed stage of treatment is completed, such information is stored in the patient database 116. In this way, the progress and the status of the treatment for a patient may be monitored and tracked.

Returning to block 1004, if a current session for the patient is not the first treatment, then the GUI prompts the operator to input whether or not the patient is responding well to the previous treatment or treatments (block 1007). In an example embodiment, the operator simply inputs a selection into the GUI indicating the progress of the patient. In another example embodiment, the patient's assessment is automatically determined from information in the patient database 116. For example, physiological information and feedback information about the patient is gathered over time along with the light therapy treatments and is stored in the database 116. If the computing system 101 detects a trend in the patient information indicating an improvement, then the computing system automatically determines that the patient is responding well to the one or more previous treatments. Otherwise, the computing system 101 determines that the patient is not responding well to the one or more previous treatments.

In an example embodiment, patient information stored in the database 116 includes one or more of: the number of headaches within a time period, the perceived severity of pain associated with the headaches according to a pain scale, the number of hours of sleep without interruption, the range of motion of the neck or spine, or both, the frequency of fatigue, and the severity of fatigue according to a fatigue severity scale. Other information about the information of the patient may be collected and stored in the database 116 and used for automatically determining the progress of the patient.

Continuing with FIG. 10, if it is determined that the patient is responding well (e.g. by user input or automatically based on patient information, or both), then the treatment protocol module 119 makes a decision to repeat the last treatment stage of the patient (block 1008). This last treatment stage associated with the patient is obtained from the patient database 116. Continuing from block 1008, the module 119 prescribes the obtained protocol for treatment (e.g. the same stage of the protocol used in the most recent previous treatment). The details, such as the light treatment device settings and the corresponding positions or areas on the body, are obtained from the protocol and area library 117. The settings are then sent to the controller interface 1001 to activate the array 1002 and the laser probe 1003 in a sequence according to the protocol. In other words, the operations of blocks 1013, 1014, 1015, 1016 and 1017 are executed, but specific to the obtained protocol.

If at block 1007, the computing system 101 determines that the patient is not responding well to the previous treatment or treatments, then the GUI is used to prompt the operator to clarify whether patient is at least one of “not improving”, “worsening” or “clinical plateau” (block 1009). If one of such conditions is true, then the treatment protocol module 119 obtains the stage of treatment last used on the patient and modifies or changes the stage of treatment (block 1010). In other words, a new stage of treatment is selected from the protocol and area library 117, which becomes the protocol automatically prescribed by the computing system 101 for the current treatment session (block 1006). The details, such as the light treatment device settings and the corresponding positions or areas on the body, are obtained from the protocol and area library 117. The settings are then sent to the controller interface 1001 to activate the array 1002 and the laser probe 1003 in a sequence according to the obtained protocol. In other words, the operations of blocks 1013, 1014, 1015, 1016 and 1017 are executed, but specific to the obtained protocol.

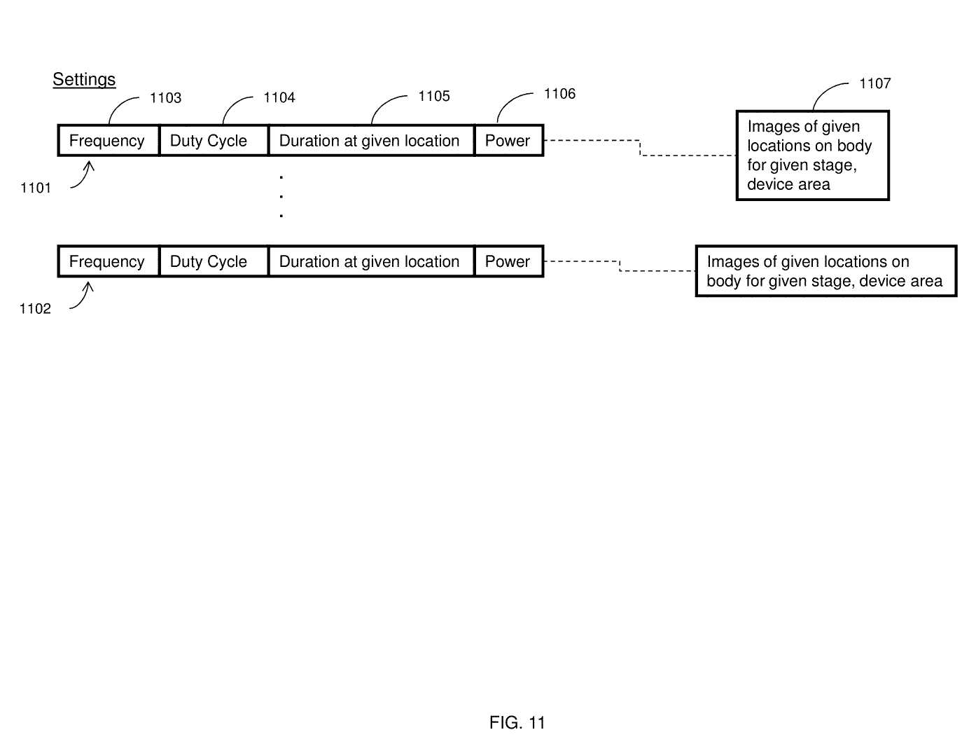

Turning to FIG. 11, examples of settings are stored in the protocols and areas library 117. Each settings entry 1101, 1102 is associated with a step in a stage. As noted above, each protocol is associated with a number of stages, and within each stage are one or more steps. Furthermore, each step is associated with one or more images which can be displayed using the display device 114 to show where to place the light treatment device. Therefore, as shown in FIG. 11, a settings entry 1101 includes a frequency setting 1103, a duty cycle setting 1104, a duration setting 1105 associated with a given position within a given area (e.g. Area 1, Area 2, Area 3), and a power setting 1106. Where there are multiple positions or locations associated with the setting entry 1101, then there are multiple duration settings 1105 corresponding to the multiple positions. The setting entry 1101 is also associated with one or more images of the one or more given positions on the body, associated with the given step. Understandably, if there are multiple positions within a given step to place the light treatment device, then there are multiple images that will be associated with the settings entry 1101.

In an example embodiment, the computing system 101 is configured to: select or adjust a frequency setting 1103 in the range of 1 Hz to 10,000 Hz; select or adjust a duty cycle setting 1104 in the range of 1% to 99%; select or adjust a duration setting 1105 associated with a given position within a given area (e.g. Area 1, Area 2, Area 3) in the range of 1 minute to 60 minutes; and select or adjust a power setting 1106 in the range of 1% to 100%. It will be appreciated that the ranges provided above are for an example only, and that other ranges may be used according to other example embodiments.

It will be appreciated that there can be different types of settings for different types of light treatment devices. For example, a laser probe does not emit light at a frequency, but as a continuous wave and at a certain power level.

In an example embodiment, the computing system selects the power setting as a percentage of a maximum power of a given light treatment device. In an example embodiment, a maximum power level for a IR laser probe is 180 mW. In an example embodiment, a maximum power level for a red laser probe is 75 mW. In an example embodiment, a maximum power level density for a red light array is 10 mW/cm². In an example embodiment, a maximum power level density for an IR light array is 20 mW/cm². These maximum values are just for example, and other maximum power levels for the various types of light treatment devices are also applicable to the principles described herein.

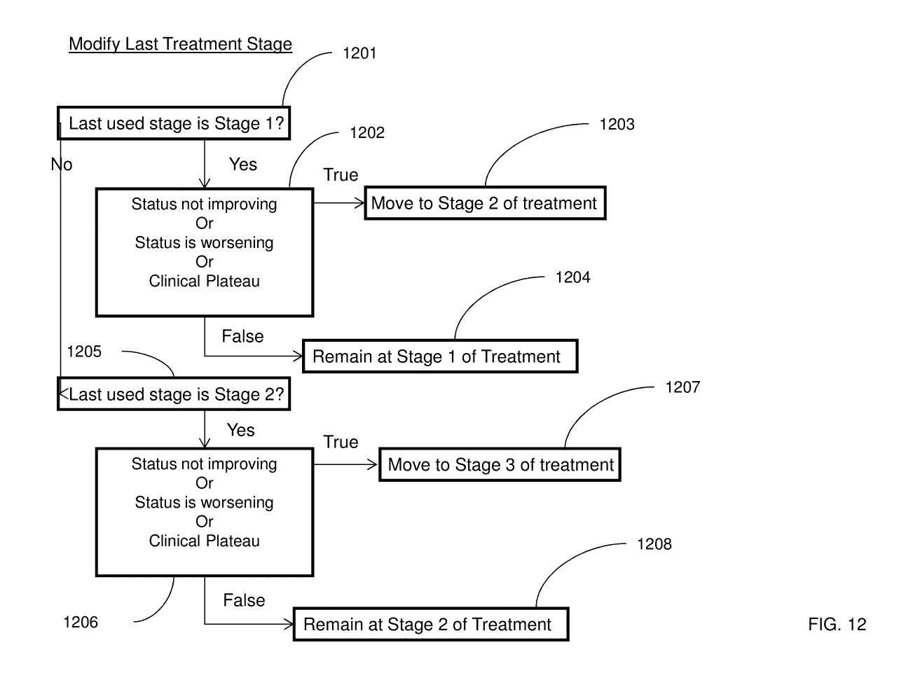

FIG. 12 shows example computer executable instructions describing blocks 1007, 1008, 1009 and 1010 in more detail for modifying the last used treatment stage and determining a new treatment stage. In particular, if the last used stage is Stage 1 (block 1201), then the computing device determines whether one of the following conditions are true: status not improving, status is worsening, or clinical plateau (block 1202). If one of such conditions is true, then the computing system 101 determines to move to Stage 2 within the protocol (block 1203). If all the conditions are false, then the computing system remains at Stage 1 of the treatment protocol (block 1204).

If the last used stage is not Stage 1, but Stage 2 (block 1205), then the computing device determines whether one of the following conditions are true: status not improving, status is worsening, or clinical plateau (block 1206). If one of such conditions is true, then the computing system 101 determines to move to Stage 3 within the protocol (block 1207). If all the conditions are false, then the computing system remains at Stage 2 of the treatment protocol (block 1208).

Turning to FIG. 13, an example embodiment of data in a protocols and areas library 117 is shown. In particular, there are number of different protocols 1301, 1305, 1309, 1313 and 1317.

Protocol 1301 corresponds to Area 1 with the classification acute labile. Protocol 1305 corresponds to Area 1 with the classification of acute stable. Protocol 1317 corresponds to Area 1 with the classification of chronic stable. Protocol 1309 corresponds to Area 2. Protocol 1313 corresponds to Area 3.

Within each protocol are a number of stages, and within each stage are a number of steps. Associated with each step are one or more positions, some of which are illustrated in FIGS. 15 to 18.

For example, in protocol 1301, there is Stage 1 (1302), Stage 2 (1303) and Stage 3 (1304). As noted above, a patient's treatment process moves from Stage 1 to Stage 2, or from Stage 2 to Stage 3, if the patient is not responding well to treatment. In other words, within protocol 1301, a patient moves between stages in sequential order.

Within Stage 1 (1302) of protocol 1301, there are three steps which are performed in sequential order. The first step is to activate the red light array device on the patient according to settings specific to that first step. The second step is to activate the IR light array device on the patient according to settings specific to that second step. The third step is to activate the red laser probe according to settings specific to that third step.

Similarly, Stage 2 (1303) of protocol 1301 includes four sequential steps respectively associated with: a red light and settings, an IR light and settings, a red laser and settings, and an IR laser and settings. Stage 3 (1304) of protocol 1301 also includes four steps.

It will be appreciated that although the same light treatment devices are used amongst different stages and different protocols, the settings of the same devices may be different. It will also be appreciated that different stages may have different numbers of steps.

As seen in FIG. 13, protocol 1305 for Area 1 and acute stable includes Stage 1 (1306), Stage 2 (1307) and Stage 3 (1308). Protocol 1317 for Area 1 and chronic stable includes Stage 1 (1318), Stage 2 (1319) and Stage 3 (1320). Protocol 1309 for Area 2 includes Stage 1 (1310), Stage 2 (1311) and Stage 3 (1312). Protocol 1313 for Area 3 includes Stage 1 (1314), Stage 2 (1315) and Stage 3 (1316).

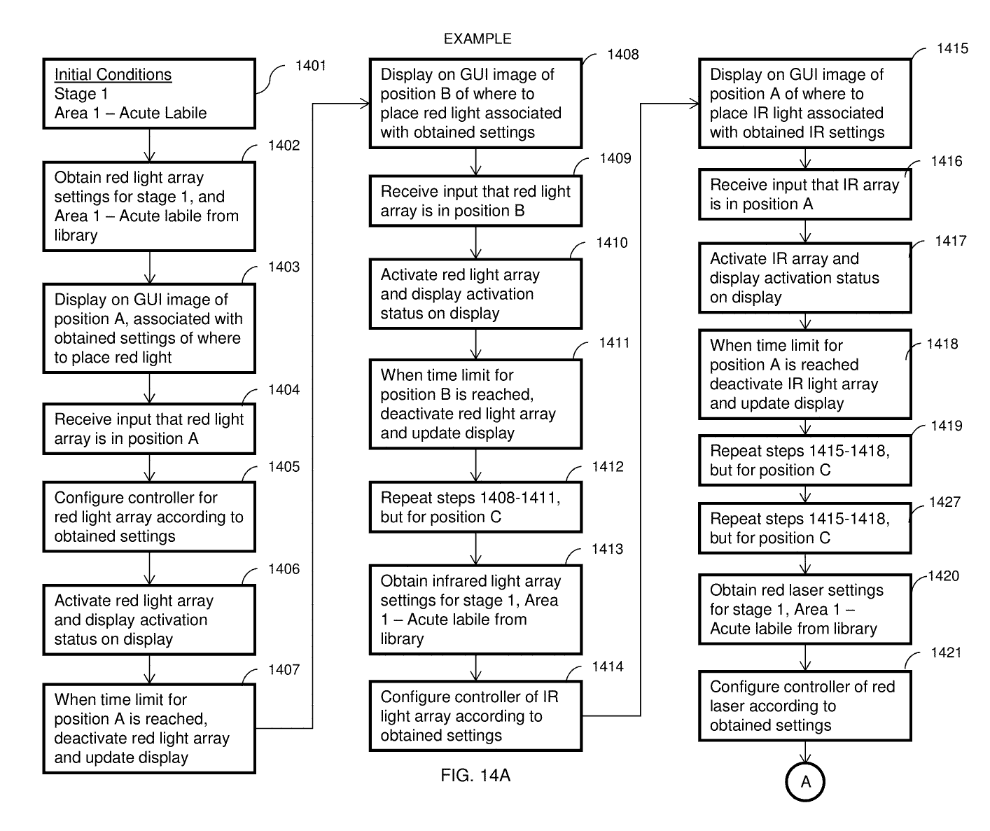

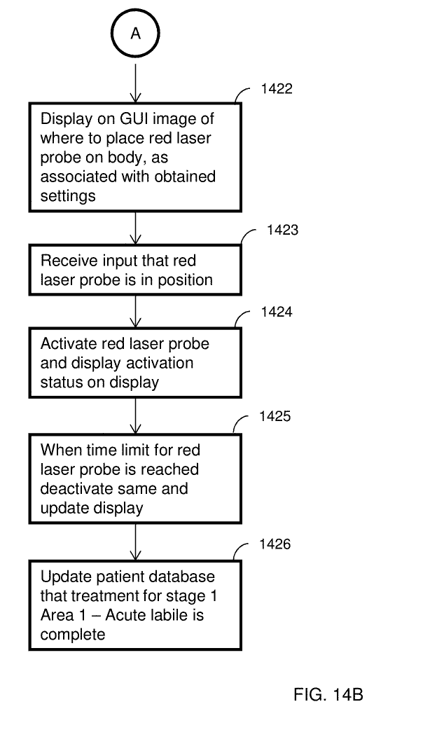

Turning to FIGS. 14A and 14B, example computer executable instructions are performed by the computing system 101 in which the example initial conditions 1401 include performing a light therapy treatment at Stage 1 (1302) for Area 1, with a concussion classified as acute labile. The executable instructions are to be used with light treatment devices having a form factor or similar to device 109, 110 or 202.

At block 1402, the computing system obtains from the library 117 the red light array settings for Stage 1, Area 1—acute labile. The settings for the red light at Stage 1 include three positions, herein called position A, position B and position C. At block 1403, the computing system displays on the GUI an image of position A. The image of position A is associated with the obtained settings. The image is used by the operator to guide where to place the red light treatment array device on the patient. After the red light treatment array device is at position A, the computing system receives an input from the operator that the red light array is in position A (block 1404). The computing system then configures the interface controller (105 or 204) for the red light array according to the obtained settings (block 1405). At block 1406, the computing system activates the red light array and displays the activation status on the display. The remaining time for the certain step can be displayed on the GUI. At block 1407, after the time limit for position A has been reached, the computing system deactivates the red light array and updates the display.

At block 1408, the computing system displays on the GUI an image of position B of where to place the red light on the patient's body, and this image is obtained from the library 117. The operator uses this image as a guide reposition the red light array. At block 1409, the computing system receives an input that the red light array is in position B. At block 1410, the computing system activates the red light array and displays the activation status on the display. At block 1411, after the time limit is reached, the computing system deactivates the red light array and updates the display device.

At block 1412, steps similar to blocks 1408-1411 are repeated, but for position C.

After using the red light, the IR light is used to treat the patient. At block 1414, the computing system obtains the infrared light array settings for Stage 1 (1302) from the library 117. The computing system then displays using the GUI an image of position A of where to position the IR light array on the patient's body (block 1415). The operator uses the image to guide the placement of the IR light array on position A. At block 1416, the computing system receives an input that the IR light array is in position A. At block 1417, the computing system activates the IR light array and displays activation status on the display device. At block 1418, after the time limit for position A is reached, the computing system deactivates the IR light array and updates the display device.

At block 1419, steps similar to blocks 1415-1418 are repeated for the IR light array, but for position B. At block 1427, following block 1419, steps similar to blocks 1415-1418 are repeated for the IR light array, but for position C.

After applying the IR light array, the computing system obtains red laser settings for Stage 1 (1302) from the library 117. At block 1421, the computing system configures the controller interface (e.g. 107 or 205) for the red laser probe according to the obtained settings. The process from FIG. 14A continues to FIG. 14B as shown by the circle containing the letter “A”.

At block 1422, the computing system displays through the GUI an image of where to place the red laser probe on the body. There are multiple positions associated with step referred to as “accupoints”. The computing system receives an input that the red laser probe is in place in the given position (block 1423) and the computing system activates the red laser probe (block 1424). The activation status of the red laser probe is displayed on the GUI. At block 1425, after the time limit for the red laser probe is reached for the given position, then the computing system deactivates the red laser probe. The display device is accordingly updated. At block 1426, the patient database is updated to reflect that a session of Stage 1 treatment is complete for the subject patient.

FIGS. 15-18 show example images and tags associated with such images that are stored in the library 117. The computing system uses the tags to identify the correct image to show in the GUI to assist the operator in placing the light array or laser probe.

Turning to FIG. 15, the tags 1501 and 1502 are associated with each of the images 1503, 1504 and 1505. The tag 1501 is Area 1 the tag 1502 is acute labile (Stage 1 or Stage 2). In other words, when a condition in which both tags 1501 and 1502 apply, then the images 1503, 1504 and 1505 are used. The images 1503, 1504 and 1505 respectively show where to place the light array on a person according position A, position B and position C. Image 1503 includes an outline of a person's body from a back perspective and an outline of a light treatment array device being positioned vertically on the upper spine. Image 1504 is an outline of a person's body from a left side view and shows an outline of a light treatment array device being positioned on the left oblique area of a person's neck. Image 1505 is an outline of a person's body from a right side view and shows an outline of a light treatment array device being positioned on the right oblique area of a person's neck.

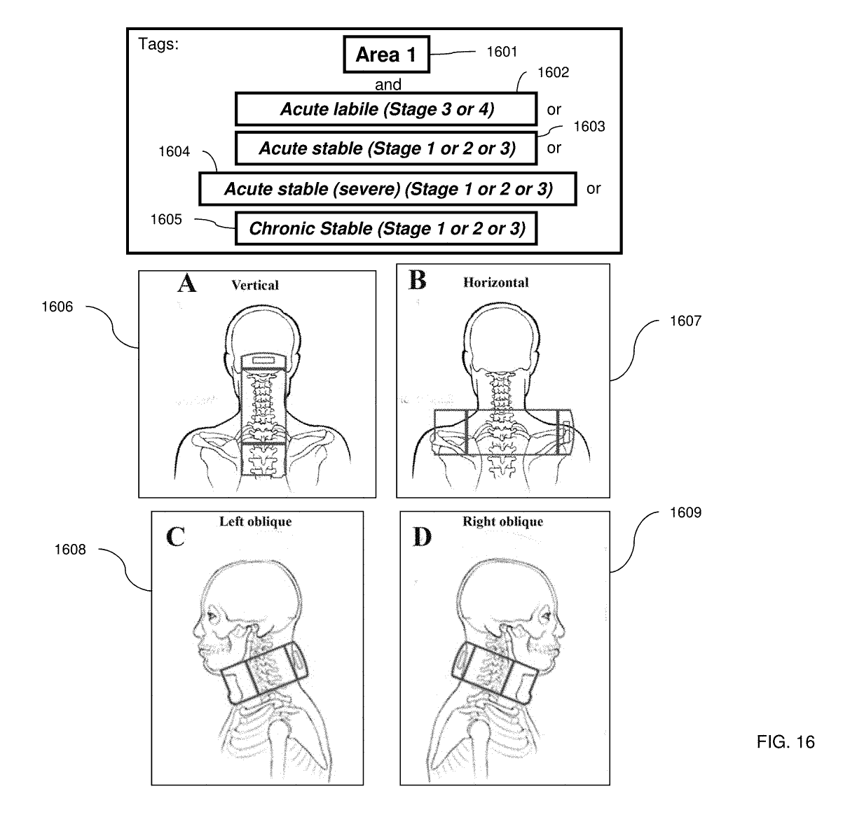

Turning to FIG. 16, tag 1601 is Area 1; tag 1602 is acute labile (Stage 3 or Stage 4); tag 1603 is acute stable (Stage 1 or Stage 2 or Stage 3); tag 1604 is acute stable (Stage 1 or Stage 2 or Stage 3); and tag 1605 is chronic stable (Stage 1 or Stage 2 or Stage 3). When a condition in which tag 1601 applies and any one of tags 1602, 1603, 104 or 1605 applies, then the images 1606, 1607, 1608 and 1609 are obtained from the library 117 and displayed on the display device 114. Image 1606 refers to position A, image 1607 refers to position B, image 1608 refers to position C, and image 1609 refers to position D.

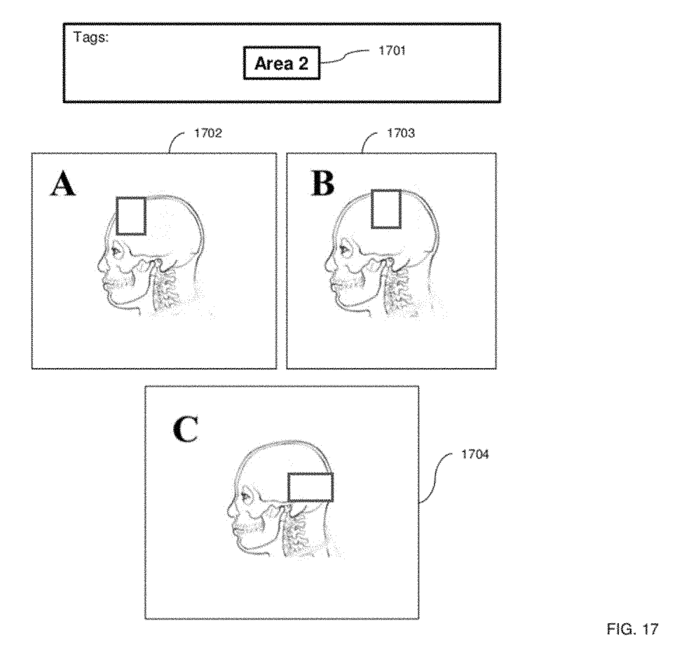

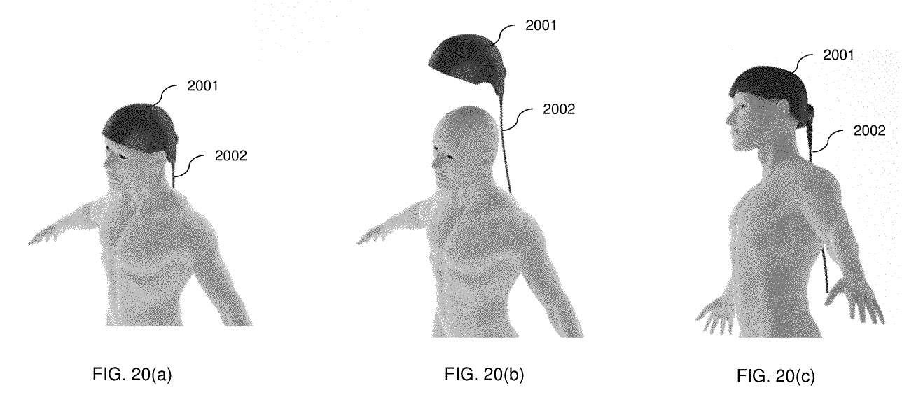

Turning to FIG. 17, tag 1701 is Area 2. When a condition in which tag 1701 applies, then the images 1702, 1703 and 1704 are obtained from the library 117 and are displayed on the display device 114. Images 1702, 1703 and 1704 respectively refer to positions A, B and C. In one example embodiment, a special treatment array that is connectable and controllable to the computing system, looks like a helmet or a baseball cap that includes the areas 1702, 1703, 1704 altogether. Therefore, when a condition in which tag 1701 applies then the image of the helmet or the baseball cap will be shown and depending on settings entry 1101, 1102 that are associated with a step in a stage, images 1702, 1703, and 1704 will be highlighted accordingly. For example, turning briefly to FIGS. 20(a), 20(b) and 20(c), a helmet 2001 is shown from different views. For example, the helmet has a wire or cable 2002 that is connectable to the computing system. On the inner surface of the helmet 2001, there are three or more sub-sections that are configured to surround a head of a person, and each of the sub-sections respectively include different groups of lights positioned at different sections on the helmet. For example, there are multiple sections that each respectively cover the areas 1702, 1703 and 1704. These different groups of lights are independently controllable by the computing system to shine the light on to different respective areas on the head according to each step in the protocol. These lights, for example, include both red light and IR light.

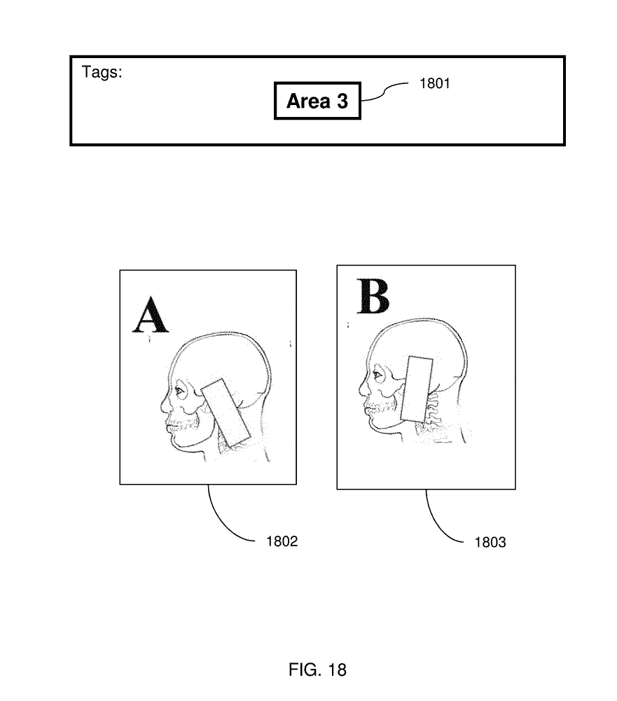

Turning to FIG. 18, tag 1801 is Area 3. When a condition in which tag 1801 applies, then the images 1802 and 1803 are obtained from the library 117 and are displayed on the display device 114. Images 1802 and 1803 respectively refer to positions A and B.

In one embodiment, images 1802 and 1803 may be replaced by FIG. 9D and instruct the user to do laser probe application on area 912.

It will be appreciated that there may be other images associated with the placement of the laser probes according to acupoints.

It will also be appreciated that if the neck and shoulder array device 701 is used, a different image is used for certain protocols, especially those related to Area 1. The instructions executed by the computing system 101 are also different when using the neck and shoulder array device 701. In one embodiment, the neck and shoulder array device 701 may be made of two separate (i.e. a top part and a bottom part) devices. Therefore, the instructions executed by the computing system 101 are also different for the top part of the device 701 (e.g. section 706)and the bottom part of the device 702 (e.g. section 707).

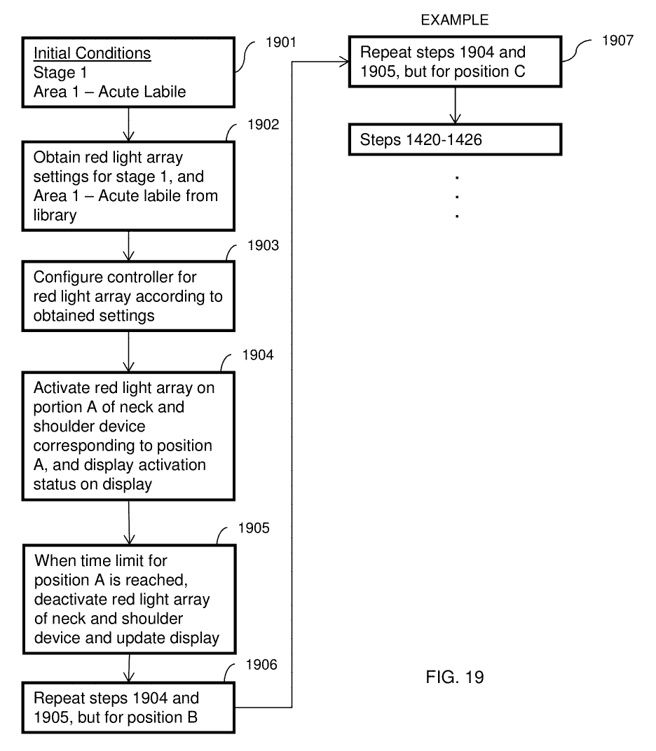

In an example embodiment of using the device 701, as per FIG. 19, the initial conditions are that treatment is for Stage 1 (1302), according to the protocol (1301) for Area 1 and the classification acute labile (block 1901). At block 1902, the computing system obtains the red light array settings for Stage 1 (1302) from the library 117. The computing system 101 configures the controller interface for the red light array according to the obtained settings (block 1903). At block 1904, the computing system activates the red light group of light emitting elements on the device 701 which correspond to the location of position A. The activation status is displayed on the GUI. At block 1905, after the time limit is reached for position A, the red light group of light emitting elements are deactivated, and the GUI is updated accordingly.

Without moving the device 701 from the patient, steps that are similar to steps 1904 and 1905 are repeated but for position B (block 1906). After block 1906, steps that are similar to steps 1904 and 1905 are repeated, but for position C (block 1907). The device 701 can remain in place since only different groups of light emitting elements on the device need to be activated and deactivated.

Steps 1420 to 1426 are then performed in relation to the laser probe red laser probe.

The above approach has been effective in several clinical cases of patients who have suffered from a concussion.

Although reference is made to emitting light at specific wavelengths, it will be appreciated that the spectral width of these wavelengths may vary. It will also be appreciated that emitting light at two or more wavelengths includes emitting light at as a substantially continuous spectrum, regardless of the relative intensity of any peaks present in the spectrum. In other words, a light source may emit light at wavelengths other than the specific target wavelengths. It will also be appreciated that although reference is made to colours of light, the wavelengths may be within or outside of the visible spectrum, for example, the wavelengths may be infrared wavelengths, near-infrared wavelengths, or even UV wavelengths.

Moreover, the light source can be emitting white light that includes several wavelengths in the spectrum. By way of example, white light may include several wavelengths in the visible spectrum. The white light may, for example, include all colors in the spectrum. Specific wavelengths emitted by the light source may be emitted by using wavelength-selective filters. For example, 660 nm wavelength can be generated from a white light source (or any other light source comprising light at 660 nm) and a 660 nm selective filter which allows at least a significant proportion of 660 nm wavelength light to transmit while substantially blocking the rest of the spectrum. The selective filter can be made of various suitable materials and shapes including, but not limited to, flat lenses, convex or concave lenses or even fiber optics.

Example general embodiment and example aspects of the systems and methods are provided below.

In an example embodiment, a system configured for light treatment is provided. The system includes: one or more light array devices that are flexible and emit red light and infrared light; at least one of a red laser probe and an infrared laser probe; a computing system connected to the one or more light array devices, the red laser probe and the infrared laser probe; the computing system comprising a processor, memory, a display device and a user input device; the memory comprising multiple protocols, each protocol comprising multiple stages, and each stage comprising multiple steps for using the one or more light array devices, and at least one of the red laser probe and the infrared laser probe, and the multiple steps respectively associated with multiple predetermined settings for configuring the one or more light array devices, and at least one of the red laser probe and the infrared laser probe; and the processor is configured to determine a given stage of a given protocol from memory, and automatically configure settings of the one or more light array devices and at least one of the red laser probe and the infrared laser probe in sequence according to the multiple predetermined settings respectively associated with given steps within the given stage.

In an example aspect of the system, the memory further includes digital images of a human head and neck, the digital images showing different locations of where to apply at least one of red light, infrared light, red laser and infrared laser on the human head or the neck.

In an example aspect of the system, the display device displays a given one of the digital images, and after receiving an input that at least one of the one or more light array devices, or the red laser probe, or the infrared laser probe is positioned at a given location on a patient's body corresponding to the given image, the processor activating the at least one of the one or more light array devices, or the red laser probe, or the infrared laser probe at the configured settings.

In an example aspect of the system, the predetermined settings comprise at least one of: a frequency setting, a duty cycle setting, a duration setting associated with a given location on a human body, and a power setting.

In an example aspect of the system, the memory of the computing system comprises one or more graphical user interfaces (GUIs) and the computing system is configured to at least: receive a first user input via a GUI displayed on the display device, the first user input used to determine that a given patient has previously received treatment; responsive to receiving a second user input via the GUI used to determine that progress of the given patient is not improving, the computing system automatically accesses a database stored in the memory to obtain a previously used stage in a given treatment protocol for the given patient and automatically selects a modified stage in the given treatment protocol from a protocol library stored in the memory; and automatically configure settings of the one or more light array devices and at least one of the red laser probe and the infrared laser probe according to the modified stage in the given treatment protocol.

In an example aspect of the system, the given treatment protocol includes multiple stages that have a certain order in the protocol library, including the modified stage being ordered subsequent to the previously used stage.

In an example aspect of the system, the memory of the computing system comprises one or more graphical user interfaces (GUIs) and the computing system is configured to at least: receive a first user input via a GUI displayed on the display device, the first user input used to determine that a given patient has previously received treatment; responsive to receiving a second user input via the GUI used to determine that progress of the given patient is improving, the computing system automatically accesses a database stored in the memory to obtain a previously used treatment protocol for the given patient and automatically selects the previously used treatment protocol; and automatically configure settings of the one or more light array devices and at least one of the red laser probe and the infrared laser probe according to the previously used treatment protocol.

In an example aspect of the system, the one or more light array devices comprises a head and neck array comprising a neck and head portion and an upper back portion that are electrically connected to each other, the upper back portion positioned below the neck and head portion; the head and neck portion comprising two wings on opposite ends that protrude forward and the head and the neck forming “C” or “U” shaped structure; and the head and neck array comprising different groups of lights at different locations on the neck and head portion and the upper back portion that are independently controllable by the computing system.

In another example embodiment, a kit of parts for light treatment is provided. The kit of parts includes: one or more light array devices that are flexible and emit red light and infrared light; at least one of a red laser probe and an infrared laser probe; a computing system connected to the one or more light array devices, the red laser probe and the infrared laser probe; the computing system comprising a processor, memory, a display device and a user input device; the memory comprising multiple protocols, each protocol comprising multiple stages, and each stage comprising multiple steps for using the one or more light array devices, and at least one of the red laser probe and the infrared laser probe, and the multiple steps respectively associated with multiple predetermined settings for configuring the one or more light array devices, and at least one of the red laser probe and the infrared laser probe; and the processor is configured to determine a given stage of a given protocol from memory, and automatically configure settings of the one or more light array devices and at least one of the red laser probe and the infrared laser probe in sequence according to the multiple predetermined settings respectively associated with given steps within the given stage.

In another example embodiment, a method performed by a computing system is provided. The method includes: accessing a memory device of the computing system, the memory comprising multiple protocols, each protocol comprising multiple stages, and each stage comprising multiple steps for using one or more light array devices, and at least one of a red laser probe and an infrared laser probe, and the multiple steps respectively associated with multiple predetermined settings for configuring the one or more light array devices, and at least one of the red laser probe and the infrared laser probe; and determining a given stage of a given protocol from the memory, and automatically configuring settings of the one or more light array devices and at least one of the red laser probe and the infrared laser probe in sequence according to the multiple predetermined settings respectively associated with given steps within the given stage, wherein the computing system is in data communication with the one or more light array devices and with at least one of the red laser probe and the infrared laser probe.

In an example aspect of the method, it further comprises the computer system accessing the memory to obtain digital images of a human head and neck, the digital images showing different locations of where to apply at least one of red light, infrared light, red laser and infrared laser on the human head or the neck.

In an example aspect of the method, it further comprises the computer system displaying a given one of the digital images on a display device on the computer system, and after receiving an input that at least one of the one or more light array devices, or the red laser probe, or the infrared laser probe is positioned at a given location on a patient's body corresponding to the given image, the computer system activating the at least one of the one or more light array devices, or the red laser probe, or the infrared laser probe at the configured settings.

In an example embodiment, a light array treatment apparatus is provided and it includes: a neck and head portion and an upper back portion that are electrically connected to each other, the upper back portion positioned below the neck and head portion; the head and neck portion comprising two wings on opposite ends that protrude forward and the head and the neck forming “C” or “U” shaped structure; the head and neck array comprising different groups of lights positioned at different locations on the neck and head portion and the upper back portion, the groups of lights independently controllable from each other.

In an example aspect of the apparatus, the lower back portion houses inflexible circuitry components for controlling the groups of lights.

In an example aspect of the apparatus, the inflexible circuity components include one or more of drivers, a communication interface, a control circuit, and electrical switches.

In an example aspect of the apparatus, the neck and head portion and the upper back portion are both flexible.

In an example aspect of the apparatus, the neck and head portion and the upper back portion are integrally formed together.

In an example aspect of the apparatus, the upper back portion comprises a left wing and a right wing protruding forward from a main body of the upper back portion.

It will be appreciated that different features of the example embodiments of the systems, the methods and the devices, as described herein, may be combined with each other in different ways. In other words, different modules, operations and components may be used together according to other example embodiments, although not specifically stated.

The steps or operations in the flow diagrams described herein are just for example. There may be many variations to these steps or operations without departing from the spirit of the invention or inventions. For instance, the steps may be performed in a differing order, or steps may be added, deleted, or modified.

Although the above has been described with reference to certain specific embodiments, various modifications thereof will be apparent to those skilled in the art without departing from the scope of the claims appended hereto.

What is claimed is:

1. A system configured for light treatment, the system comprising:
one or more light array devices that are flexible and emit red light and infrared light;
at least one of a red laser probe and an infrared laser probe;
a computing system connected to the one or more light array devices, the red laser probe and the infrared laser probe;
the computing system comprising a processor, memory, a display device and a user input device;
the memory comprising multiple protocols, each protocol comprising multiple stages, and each stage comprising multiple steps for using the one or more light array devices, and at least one of the red laser probe and the infrared laser probe, and the multiple steps respectively associated with multiple predetermined settings for configuring the one or more light array devices, and at least one of the red laser probe and the infrared laser probe; and
the processor is configured to determine a given stage of a given protocol from memory, and automatically configure settings of the one or more light array devices and at least one of the red laser probe and the infrared laser probe in sequence according to the multiple predetermined settings respectively associated with given steps within the given stage.

2. The system of claim 1 wherein the memory further comprises digital images of a human head and neck, the digital images showing different locations of where to apply at least one of red light, infrared light, red laser and infrared laser on the human head or the neck.

3. The system of claim 2 wherein the display device displays a given one of the digital images, and after receiving an input that at least one of the one or more light array devices, or the red laser probe, or the infrared laser probe is positioned at a given location on a patient's body corresponding to the given image, the processor activating the at least one of the one or more light array devices, or the red laser probe, or the infrared laser probe at the configured settings.

4. The system of claim 1 wherein the predetermined settings comprise at least one of: a frequency setting, a duty cycle setting, a duration setting associated with a given location on a human body, and a power setting.

5. The system of claim 1 wherein the memory of the computing system comprises one or more graphical user interfaces (GUIs) and the computing system is configured to at least:
receive a first user input via a GUI displayed on the display device, the first user input used to determine that a given patient has previously received treatment;
responsive to receiving a second user input via the GUI used to determine that progress of the given patient is not improving, the computing system automatically accesses a database stored in the memory to obtain a previously used stage in a given treatment protocol for the given patient and automatically selects a modified stage in the given treatment protocol from a protocol library stored in the memory; and
automatically configure settings of the one or more light array devices and at least one of the red laser probe and the infrared laser probe according to the modified stage in the given treatment protocol.

6. The system of claim 5 wherein the given treatment protocol includes multiple stages that have a certain order in the protocol library, including the modified stage being ordered subsequent to the previously used stage.

7. The system of claim 1 wherein the memory of the computing system comprises one or more graphical user interfaces (GUIs) and the computing system is configured to at least:
receive a first user input via a GUI displayed on the display device, the first user input used to determine that a given patient has previously received treatment;
responsive to receiving a second user input via the GUI used to determine that progress of the given patient is improving, the computing system automatically accesses a database stored in the memory to obtain a previously used treatment protocol for the given patient and automatically selects the previously used treatment protocol; and
automatically configure settings of the one or more light array devices and at least one of the red laser probe and the infrared laser probe according to the previously used treatment protocol.

8. The system of claim 1 wherein the one or more light array devices comprises a head and neck array comprising a neck and head portion and an upper back portion that are electrically connected to each other, the upper back portion positioned below the neck and head portion; the head and neck portion comprising two wings on opposite ends that protrude forward and the head and the neck forming “C” or “U” shaped structure; and the head and neck array comprising different groups of lights at different locations on the neck and head portion and the upper back portion that are independently controllable by the computing system.

9. The system of claim 8 wherein the head and neck array comprises two separate sections that are assembled together to form the head and neck array, and wherein the two separate sections are independently controllable by the computing system according to each step in the protocol.

10. The system of claim 1 wherein the one or more light array devices comprises a helmet comprising three or more sub-sections that are configured to surround a head, each of the sub-sections respectively comprising different groups of lights at positioned at different sections on the helmet and that are independently controllable by the computing system to shine the light on to different respective areas on the head according to each step in the protocol.

11. A kit of parts for light treatment, the kit of parts comprising:
one or more light array devices that are flexible and emit red light and infrared light;
at least one of a red laser probe and an infrared laser probe;
a computing system connected to the one or more light array devices, the red laser probe and the infrared laser probe;
the computing system comprising a processor, memory, a display device and a user input device;
the memory comprising multiple protocols, each protocol comprising multiple stages, and each stage comprising multiple steps for using the one or more light array devices, and at least one of the red laser probe and the infrared laser probe, and the multiple steps respectively associated with multiple predetermined settings for configuring the one or more light array devices, and at least one of the red laser probe and the infrared laser probe; and
the processor is configured to determine a given stage of a given protocol from memory, and automatically configure settings of the one or more light array devices and at least one of the red laser probe and the infrared laser probe in sequence according to the multiple predetermined settings respectively associated with given steps within the given stage.

12. The kit of parts of claim 11 wherein the memory further comprises digital images of a human head and neck, the digital images showing different locations of where to apply at least one of red light, infrared light, red laser and infrared laser on the human head or the neck.

13. The kit of parts of claim 12 wherein the display device displays a given one of the digital images, and after receiving an input that at least one of the one or more light array devices, or the red laser probe, or the infrared laser probe is positioned at a given location on a patient's body corresponding to the given image, the processor activating the at least one of the one or more light array devices, or the red laser probe, or the infrared laser probe at the configured settings.

14. The kit of parts of claim 11 wherein the predetermined settings comprise at least one of: a frequency setting, a duty cycle setting, a duration setting associated with a given location on a human body, and a power setting.

15. The kit of parts of claim 11 wherein the memory of the computing system comprises one or more graphical user interfaces (GUIs) and the computing system is configured to at least:
receive a first user input via a GUI displayed on the display device, the first user input used to determine that a given patient has previously received treatment;
responsive to receiving a second user input via the GUI used to determine that progress of the given patient is not improving, the computing system automatically accesses a database stored in the memory to obtain a previously used stage in a given treatment protocol for the given patient and automatically selects a modified stage in the given treatment protocol from a protocol library stored in the memory; and
automatically configure settings of the one or more light array devices and at least one of the red laser probe and the infrared laser probe according to the modified stage in the given treatment protocol.

16. The kit of parts of claim 15 wherein the given treatment protocol includes multiple stages that have a certain order in the protocol library, including the modified stage being ordered subsequent to the previously used stage.

17. The kit of parts of claim 11 wherein the memory of the computing system comprises one or more graphical user interfaces (GUIs) and the computing system is configured to at least:
receive a first user input via a GUI displayed on the display device, the first user input used to determine that a given patient has previously received treatment;
responsive to receiving a second user input via the GUI used to determine that progress of the given patient is improving, the computing system automatically accesses a database stored in the memory to obtain a previously used treatment protocol for the given patient and automatically selects the previously used treatment protocol; and
automatically configure settings of the one or more light array devices and at least one of the red laser probe and the infrared laser probe according to the previously used treatment protocol.

18. The kit of parts of claim 11 wherein the one or more light array devices comprises a head and neck array comprising a neck and head portion and an upper back portion that are electrically connected to each other, the upper back portion positioned below the neck and head portion; the head and neck portion comprising two wings on opposite ends that protrude forward and the head and the neck forming “C” or “U” shaped structure; and the head and neck array comprising different groups of lights at different locations on the neck and head portion and the upper back portion that are independently controllable by the computing system.

19. The kit of parts of claim 18 wherein the head and neck array comprises two separate sections that are assembled together to form the head and neck array, and wherein the two separate sections are independently controllable by the computing system according to each step in the protocol.

20. The kit of parts of claim 11 wherein the one or more light array devices comprises a helmet comprising three or more sub-sections that are configured to surround a head, each of the sub-sections respectively comprising different groups of lights at positioned at different sections on the helmet and that are independently controllable by the computing system to shine the light on to different respective areas on the head according to each step in the protocol.

21. A method performed by a computing system, the method comprising:
accessing a memory device of the computing system, the memory comprising multiple protocols, each protocol comprising multiple stages, and each stage comprising multiple steps for using one or more light array devices, and at least one of a red laser probe and an infrared laser probe, and the multiple steps respectively associated with multiple predetermined settings for configuring the one or more light array devices, and at least one of the red laser probe and the infrared laser probe; and
determining a given stage of a given protocol from the memory, and automatically configuring settings of the one or more light array devices and at least one of the red laser probe and the infrared laser probe in sequence according to the multiple predetermined settings respectively associated with given steps within the given stage, wherein the computing system is in data communication with the one or more light array devices and with at least one of the red laser probe and the infrared laser probe.

22. The method of claim 21 further comprising the computer system accessing the memory to obtain digital images of a human head and neck, the digital images showing different locations of where to apply at least one of red light, infrared light, red laser and infrared laser on the human head or the neck.

23. The method of claim 22 further comprising the computer system displaying a given one of the digital images on a display device on the computer system, and after receiving an input that at least one of the one or more light array devices, or the red laser probe, or the infrared laser probe is positioned at a given location on a patient's body corresponding to the given image, the computer system activating the at least one of the one or more light array devices, or the red laser probe, or the infrared laser probe at the configured settings.

24. A light array treatment apparatus comprising:
a neck and head portion and an upper back portion that are electrically connected to each other, the upper back portion positioned below the neck and head portion; the head and neck portion comprising two wings on opposite ends that protrude forward and the head and the neck forming “C” or “U” shaped structure; the head and neck array comprising different groups of lights positioned at different locations on the neck and head portion and the upper back portion, the groups of lights independently controllable from each other.

25. The apparatus of claim 24 wherein the head and neck array comprises two separate sections that are assembled together to form the head and neck array, and wherein the two separate sections are independently controllable by the computing system according to each step in the protocol.

26. The apparatus of claim 24, wherein the lower back portion houses inflexible circuitry components for controlling the groups of lights.

27. The apparatus of claim 26, wherein the inflexible circuity components include one or more of drivers, a communication interface, a control circuit, and electrical switches.

28. The apparatus of claim 24, wherein the neck and head portion and the upper back portion are both flexible.

29. The apparatus of claim 24, wherein the neck and head portion and the upper back portion are integrally formed together.

30. The apparatus of claim 24, wherein the upper back portion comprises a left wing and a right wing protruding forward from a main body of the upper back portion.
